(19) United States
(12) Patent Application Publication
(10) Pub. No.: US 2026/0355477 A1
(43) Pub. Date: Nov. 12, 2026

(54) Shunt Between Two Body Lumens

(71) Applicant: Nidus Biomedical, LLC, Irvine, CA (US)

(72) Inventors: Robert C. Taft, Orange, CA (US); Alexander Siegel, Aliso Viejo, CA (US); Stanton J. Rowe, Newport Coast, CA (US); Glen Rabito, Lake Forest, CA (US); Andrew May, Irvine, CA (US); Ryan Bondy, Dana Point, CA (US)

(73) Assignee: Nidus Biomedical, LLC, Irvine, CA (US)

(21) Appl. No.: 19/719,979

(22) Filed: Jun. 25, 2026

Related U.S. Application Data
(63) Continuation of application No. PCT/US2026/014801, filed on Feb. 10, 2026.
(60) Provisional application No. 63/756,631, filed on Feb. 10, 2025.
(60) Provisional application No. 63/906,147, filed on Oct. 27, 2025.

(51) Int. Cl. A61M 27/00; A61B 18/00; A61B 18/14

(52) U.S. Cl. CPC A61M 27/002; A61B 18/1492; A61B 2018/00351; A61M 2205/3331; A61M 2210/125

(57) ABSTRACT

Methods, systems, and devices are described for creating and evaluating a shunt between adjacent body lumens, including a shunt between a pulmonary vein and a left atrial appendage to augment blood flow and reduce stasis. Example devices include expandable shunt stents, hybrid and braided stent configurations, tissue-apposition clips, and delivery systems configured for transcatheter deployment. Functional assessment of the shunt may include contrast washout analysis and machine-learning-based evaluation of imaging data to characterize shunt effectiveness and associated clinical metrics.

### RELATED APPLICATIONS

This application is a continuation of International Application PCT/US2026/014801 filed Feb. 10, 2026, which claims benefit of and priority to U.S. Provisional Application Ser. No. 63/756,631 filed Feb. 10, 2025 entitled Braided Rivet Stent, and U.S. Provisional Application Ser. No. 63/906,147 filed Oct. 27, 2025 entitled Shunt Between Two Body Lumens both of which are hereby incorporated by reference in their entireties.

### BACKGROUND

The left atrial appendage (LAA) is a small pouch that typically extends from the left atrium of a heart, managing blood volume within the heart by releasing natriuretic peptides when the LAA stretches.

Some conditions, such as atrial fibrillation, may reduce blood flow into and out of the LAA, resulting in coagulation and formation of thrombi. When one or more of these thrombi migrate to the brain of a patient, blood flow to portions of the brain is occluded, resulting in a stroke.

For that reason, patients with conditions such as atrial fibrillation are typically treated with anticoagulants and often with surgical or catheter-based methods for occluding the LAA. Once the LAA is healed and fully occluded, the risk of stroke is significantly decreased, frequently eliminating the need for continued anticoagulants.

### SUMMARY

In some aspects, the techniques described herein relate to a method of creating a shunt between a left atrial appendage (LAA) and a pulmonary vein (PV), the method including: advancing a catheter-based instrument into a heart; forming an opening that traverses a wall of the left atrial appendage and a wall of the pulmonary vein; and, establishing a flow path between the pulmonary vein and the left atrial appendage that permits blood to pass into and out of the left atrial appendage.

In some aspects, the techniques described herein relate to a method, wherein advancing the catheter-based instrument includes advancing an outer sheath over a guidewire toward the left atrial appendage and orienting a distal opening of the outer sheath toward a selected pulmonary vein.

In some aspects, the techniques described herein relate to a method, wherein the outer sheath includes a pre-formed distal curve, a steerable mechanism to direct the distal opening toward the PV, or both.

In some aspects, the techniques described herein relate to a method, wherein forming the opening includes delivering radiofrequency energy via a guidewire or via an electrode at a distal tip of an inner shaft advanced through an outer sheath.

In some aspects, the techniques described herein relate to a method, wherein forming the opening includes mechanical puncture or laser energy delivery.

In some aspects, the techniques described herein relate to a method, further including apposing the LAA wall and the PV wall prior to or during formation of the opening using a support instrument advanced through a sheath.

In some aspects, the techniques described herein relate to a method, wherein establishing the flow path includes deploying a shunt stent across the opening to define a shunt lumen between the left atrial appendage and the pulmonary vein.

In some aspects, the techniques described herein relate to a method, wherein the shunt stent is self-expanding and is released by withdrawing an outer shaft from over an inner shaft on which the stent is carried.

In some aspects, the techniques described herein relate to a method, wherein the shunt stent includes a strut portion and at least one braided or woven portion that foreshortens upon expansion to engage tissue on opposite sides of the opening.

In some aspects, the techniques described herein relate to a method, wherein the strut portion is formed from a laser-cut tubular member and the braided portion includes metallic or polymeric filaments.

In some aspects, the techniques described herein relate to a method, wherein the shunt stent is a fully braided construct having a smaller-diameter middle portion and enlarged end portions that provide rivet-like tissue engagement.

In some aspects, the techniques described herein relate to a method, further including crimping the end portions with localized hinges positioned along end loops to permit acute-angle bending during loading onto a delivery device and elastic recovery upon deployment.

In some aspects, the techniques described herein relate to a method, wherein each hinge includes tubular end segments attached to cut ends of a braid wire and a middle compliant region formed by a laser-cut cut-away or groove pattern.

In some aspects, the techniques described herein relate to a method, wherein the hinge provides a bend angle between about 30 degrees and about 180 degrees during crimping and elastic recovery to within about 10 degrees of a pre-crimp orientation.

In some aspects, the techniques described herein relate to a method, further including performing a contrast washout assessment of the LAA after establishing the flow path to evaluate shunt effectiveness.

In some aspects, the techniques described herein relate to a method of assessing effectiveness of a shunt between a pulmonary vein (PV) and a left atrial appendage (LAA) in a patient, the method including: introducing radiographic contrast into the LAA; acquiring a time-resolved image sequence using an imaging modality configured to generate time-resolved images, the image sequence depicting washout of the contrast from the LAA over a plurality of cardiac cycles; determining, from the image sequence, one or more washout metrics indicative of clearance of the contrast from the LAA; and generating, based on the one or more washout metrics, an assessment of shunt effectiveness.

In some aspects, the techniques described herein relate to a method, wherein the one or more washout metrics include a number of cardiac cycles required for substantial or complete clearance of contrast from the LAA.

In some aspects, the techniques described herein relate to a method, further including identifying one or more regions of persistent contrast retention within the LAA from the image sequence, and wherein the assessment of shunt effectiveness is generated in view of the identified regions.

In some aspects, the techniques described herein relate to a method, further including evaluating directional flow patterns within the LAA induced by the shunt and incorporating the directional flow patterns into the assessment.

In some aspects, the techniques described herein relate to a method, further including comparing the one or more washout metrics to pre-shunt baseline values associated with the patient.

In some aspects, the techniques described herein relate to a method, further including comparing the one or more washout metrics to population-derived reference values.

In some aspects, the techniques described herein relate to a method, wherein acquiring the time-resolved fluoroscopic image sequence includes fluoroscopic imaging during and after injection of contrast into the LAA.

In some aspects, the techniques described herein relate to a method, wherein generating the assessment of shunt effectiveness includes generating a quantitative score or a categorical classification indicative of improved LAA circulation.

In some aspects, the techniques described herein relate to a method, further including predicting a patient stroke risk based at least in part on the one or more washout metrics.

In some aspects, the techniques described herein relate to a method, wherein the shunt permits pulmonary venous blood to flow into and out of the LAA to augment circulation within the LAA.

In some aspects, the techniques described herein relate to a computer-implemented method of training a machine-learning model to evaluate contrast washout characteristics of a left atrial appendage (LAA) following creation of a shunt between a pulmonary vein (PV) and the LAA, the method including: obtaining a training dataset including time-resolved fluoroscopic image sequences from a plurality of subjects, each image sequence depicting injection of contrast into the LAA and subsequent washout over multiple cardiac cycles; receiving, for the image sequences, reference indicators of washout behavior including one or more of washout speed, contrast persistence, or directional flow patterns; preprocessing the image sequences to generate model-ready inputs; and training the machine-learning model on the model-ready inputs and the reference indicators to learn a mapping between contrast-dissipation features and LAA washout characteristics associated with shunt effectiveness.

In some aspects, the techniques described herein relate to a method, wherein preprocessing includes one or more of temporal normalization, segmentation of an LAA region, motion compensation, or extraction of pixel-intensity time curves representing contrast clearance.

In some aspects, the techniques described herein relate to a method, wherein training the machine-learning model includes training a convolutional neural network, a recurrent neural network, a transformer-based architecture, or combinations thereof.

In some aspects, the techniques described herein relate to a method, further including incorporating patient stroke outcome data associated with at least a subset of the subjects, such that the model is trained to associate washout characteristics with stroke risk.

In some aspects, the techniques described herein relate to a method, further including validating the trained machine-learning model using one or more held-out image sequences.

In some aspects, the techniques described herein relate to a method, wherein the trained machine-learning model is configured to output a quantitative score, a categorical classification, or a visualization highlighting regions of delayed washout or improved flow.

In some aspects, the techniques described herein relate to a method, further including updating the trained machine-learning model with additional patient data using adaptive learning.

In some aspects, the techniques described herein relate to a method, wherein the training dataset includes image sequences acquired following formation of the shunt between the PV and the LAA.

In some aspects, the techniques described herein relate to a method, wherein the reference indicators include at least one of: (i) a number of cardiac cycles required for substantial or complete clearance of contrast from the LAA; (ii) identification of regions of persistent contrast retention; (iii) directional flow patterns induced by the shunt; and/or (iv) elapsed time.

In some aspects, the techniques described herein relate to a method, wherein outputs generated by the trained machine-learning model are compared with subject-specific baseline values or population-derived reference ranges.

In some aspects, the techniques described herein relate to a method, further including storing the trained machine-learning model in a non-transitory computer-readable medium for subsequent assessment of shunt effectiveness in additional patients.

In some aspects, the techniques described herein relate to a clip configured to maintain apposition between a wall of a left atrial appendage (LAA) and a wall of a pulmonary vein, the clip including: an elongate body having a first end region, a second end region, and a curved middle portion extending between the first and second end regions; wherein the first end region is configured to engage tissue on a first side of an opening between the LAA and the pulmonary vein; wherein the second end region is configured to engage tissue on a second side of the opening; and wherein the curved middle portion is shaped to bias the first and second end regions toward one another to maintain apposition of the LAA wall and the pulmonary vein wall.

In some aspects, the techniques described herein relate to a clip, wherein the first end region and the second end region each include an enlarged tissue-engaging structure presenting a bearing surface configured to distribute contact forces against the tissue.

In some aspects, the techniques described herein relate to a clip, wherein at least one of the enlarged tissue-engaging structures includes a closed loop, an open loop, a teardrop shape, a D-shape, a rounded paddle, a Y-shape, or a multi-filament frame.

In some aspects, the techniques described herein relate to a clip, wherein the curved middle portion has a resting curvature selected to conform to native atrial anatomy and to apply a biasing force that draws the LAA wall toward the pulmonary vein wall.

In some aspects, the techniques described herein relate to a clip, wherein the curved middle portion is asymmetrically curved between the first end region and the second end region.

In some aspects, the techniques described herein relate to a clip, wherein at least one of the first or second end regions defines an opening through which a shunt-forming instrument may be advanced while the clip maintains tissue apposition.

In some aspects, the techniques described herein relate to a clip, wherein the clip is formed from an elastic or superelastic material such that the clip is deformable into a constrained delivery configuration and recoverable to an expanded clamping configuration upon deployment.

In some aspects, the techniques described herein relate to a clip, wherein the elastic or superelastic material includes nitinol.

In some aspects, the techniques described herein relate to a clip, wherein the clip is configured for transcatheter delivery through a delivery catheter.

In some aspects, the techniques described herein relate to a clip, wherein the clip is configured to be temporarily deployed and subsequently removed after shunt formation.

In some aspects, the techniques described herein relate to a shunt stent configured to establish a flow path between adjacent body lumens, the shunt stent including: a strut portion defining a central shunt lumen; and at least one tissue-engaging portion coupled to the strut portion, wherein the strut portion is formed from a laser-cut tubular member and provides radial strength, and wherein the tissue-engaging portion is configured to radially expand and longitudinally foreshorten to engage tissue.

In some aspects, the techniques described herein relate to a shunt stent, wherein the tissue-engaging portion includes a braided or woven structure formed from one or more filaments.

In some aspects, the techniques described herein relate to a shunt stent, wherein tissue-engaging portions are positioned on opposing sides of the strut portion to form a rivet-like configuration.

In some aspects, the techniques described herein relate to a shunt stent, further including a semi-porous or non-porous covering along at least a portion of the shunt stent.

In some aspects, the techniques described herein relate to a shunt stent formed from a single laser-cut tubular member, including: a strut portion defining a central shunt lumen; and a plurality of free-ended struts extending from the strut portion and configured to be formed into tissue-engaging portions that foreshorten upon deployment.

In some aspects, the techniques described herein relate to a shunt stent, wherein the free-ended struts are woven or braided together after laser cutting.

In some aspects, the techniques described herein relate to a shunt stent, wherein the strut portion and the tissue-engaging portions define a continuous load path.

In some aspects, the techniques described herein relate to a shunt stent, wherein the tissue-engaging portions form a rivet-like configuration when deployed.

In some aspects, the techniques described herein relate to a shunt stent configured to establish a flow path between adjacent body lumens, the shunt stent including: a single tubular member formed from a metallic tube and laser-cut to define: a strut portion arranged to form a central shunt lumen; and a plurality of free-ended struts extending from opposing longitudinal ends of the strut portion, wherein the free-ended struts are configured to be formed into tissue-engaging portions that radially expand and longitudinally foreshorten upon deployment to engage tissue on opposite sides of an opening between the adjacent body lumens.

In some aspects, the techniques described herein relate to a shunt stent, wherein the strut portion and the free-ended struts are integrally formed from the single tubular member such that the shunt stent defines a continuous load path without mechanically joined components.

In some aspects, the techniques described herein relate to a shunt stent, wherein the free-ended struts have distal ends that are not secured to the strut portion after laser cutting.

In some aspects, the techniques described herein relate to a shunt stent, wherein the free-ended struts are woven, braided, interlaced, or otherwise formed together after laser cutting to define the tissue-engaging portions.

In some aspects, the techniques described herein relate to a shunt stent, wherein the tissue-engaging portions are shape-set such that, upon release from a constrained delivery configuration, the tissue-engaging portions radially expand and longitudinally retract relative to the strut portion.

In some aspects, the techniques described herein relate to a shunt stent, wherein the tissue-engaging portions form enlarged regions relative to the central shunt lumen to produce a rivet-like configuration when deployed.

In some aspects, the techniques described herein relate to a shunt stent, wherein the strut portion is configured to establish and maintain a target diameter of the central shunt lumen while the tissue-engaging portions conform to surrounding tissue.

In some aspects, the techniques described herein relate to a shunt stent, wherein longitudinal foreshortening of the tissue-engaging portions applies compressive forces to tissue captured between the tissue-engaging portions on opposing sides of the opening.

In some aspects, the techniques described herein relate to a shunt stent, wherein the tissue-engaging portions assume concave, flared, or trumpet-shaped profiles when deployed.

In some aspects, the techniques described herein relate to a shunt stent, wherein the strut portion includes one or more rows of repeating cell geometries selected from diamond-shaped, rhomboid, hexagonal, or circular shapes.

In some aspects, the techniques described herein relate to a shunt stent, wherein the free-ended struts originate from terminal cells of the strut portion.

In some aspects, the techniques described herein relate to a shunt stent, further including a semi-porous or non-porous layer disposed along at least a portion of the strut portion to reduce leakage around the central shunt lumen.

In some aspects, the techniques described herein relate to a shunt stent, wherein the layer includes ePTFE, polyurethane, silicone, electro spun materials, or a polymer-coated textile material.

In some aspects, the techniques described herein relate to a shunt stent, wherein the shunt stent is configured to be delivered in a radially constrained configuration on a delivery shaft and deployed by withdrawal of an outer sheath.

In some aspects, the techniques described herein relate to a shunt stent, wherein the adjacent body lumens include a pulmonary vein and a left atrial appendage.

In some aspects, the techniques described herein relate to a shunt stent configured to establish a flow path between adjacent body lumens, the shunt stent including: a braided tubular structure formed from one or a plurality of elongate filaments that define a central shunt lumen; and at least one locking mechanism. wherein the locking mechanism is configured to selectively resist relative axial movement between the filaments when the braided tubular structure reaches a target expanded diameter.

In some aspects, the techniques described herein relate to a shunt stent, wherein the at least one locking mechanism is positioned at a crossover between portions of the one or a plurality of filaments of the braided tubular structure; the locking mechanism includes a sleeve mounted on a first filament of the braided tubular structure.

In some aspects, the techniques described herein relate to a shunt stent, wherein the sleeve defines a groove oriented at an angle relative to a longitudinal axis of the first filament.

In some aspects, the techniques described herein relate to a shunt stent, wherein a second filament crossing the first filament is configured to seat within the groove when the braided tubular structure reaches the target expanded diameter.

In some aspects, the techniques described herein relate to a shunt stent, wherein seating of the second filament within the groove inhibits further relative sliding between the first and second filaments at the crossover.

In some aspects, the techniques described herein relate to a shunt stent, wherein the groove has a depth and profile selected to permit locking at the target expanded diameter while allowing relative filament movement at diameters smaller than the target expanded diameter.

In some aspects, the techniques described herein relate to a shunt stent, wherein the sleeve is a tubular member formed from a metallic hypotube or microtube.

In some aspects, the techniques described herein relate to a shunt stent, wherein the sleeve is radiopaque or includes a radiopaque marker to permit visualization of locking under fluoroscopy.

In some aspects, the techniques described herein relate to a shunt stent, wherein the locking mechanism is configured as a permanent lock that resists disengagement under physiologic loading.

In some aspects, the techniques described herein relate to a shunt stent, wherein the locking mechanism is configured to be overridable such that application of a predetermined axial force, torsional force, or radial expansion force permits disengagement of the locking mechanism.

In some aspects, the techniques described herein relate to a shunt stent, wherein a plurality of locking mechanisms are distributed circumferentially around the braided tubular structure.

In some aspects, the techniques described herein relate to a shunt stent, wherein the locking mechanisms are positioned in one or more longitudinal rows between proximal and distal ends of the shunt stent.

In some aspects, the techniques described herein relate to a shunt stent, wherein the locking mechanisms are positioned in a middle portion of the braided tubular structure to stabilize a lumen-defining region of the shunt stent.

In some aspects, the techniques described herein relate to a shunt stent, wherein the locking mechanisms are positioned in enlarged end portions of the braided tubular structure to stabilize tissue-engaging regions.

In some aspects, the techniques described herein relate to a shunt stent, wherein the locking mechanism includes a first sleeve mounted on a first filament and a second sleeve mounted on a second filament crossing the first filament.

In some aspects, the techniques described herein relate to a shunt stent, wherein the first sleeve and the second sleeve each define a groove, and the grooves are configured to interlock with one another when the braided tubular structure reaches the target expanded diameter.

In some aspects, the techniques described herein relate to a shunt stent, wherein interlocking of the grooves inhibits both axial displacement and rotational movement between the first and second filaments.

In some aspects, the techniques described herein relate to a shunt stent, wherein the grooves have complementary non-linear or jagged profiles that mechanically engage when aligned.

In some aspects, the techniques described herein relate to a shunt stent, wherein a dual-sleeve locking mechanism provides increased engagement area relative to a single-sleeve locking mechanism.

In some aspects, the techniques described herein relate to a shunt stent, wherein the locking mechanism is mounted on at least one filament prior to braiding of the braided tubular structure.

In some aspects, the techniques described herein relate to a shunt stent, wherein the braided tubular structure is heat-set after assembly such that the locking mechanism engages at the target expanded diameter.

In some aspects, the techniques described herein relate to a shunt stent, wherein the locking mechanism stabilizes the central shunt lumen at the target expanded diameter while permitting limited compliance of the braided tubular structure under physiologic loading.

In some aspects, the techniques described herein relate to a shunt stent configured to establish a flow path between adjacent body lumens, the shunt stent including: a braided tubular structure formed from a plurality of elongate filaments defining a central shunt lumen and at least one enlarged end portion; and one or more hinges positioned along the braided tubular structure at locations associated with the enlarged end portion, wherein each hinge provides localized bending compliance that permits controlled articulation of the braided tubular structure during radial constraining of the shunt stent for delivery, while allowing elastic recovery toward an expanded configuration upon deployment.

In some aspects, the techniques described herein relate to a shunt stent, wherein at least one hinge includes a tubular member having a compliant region configured to preferentially bend relative to adjacent portions of the braided tubular structure.

In some aspects, the techniques described herein relate to a shunt stent, wherein the tubular member is formed from a metallic hypotube or microtube.

In some aspects, the techniques described herein relate to a shunt stent, wherein the compliant region includes a cut-away window, slot, groove, or laser-cut pattern formed in a wall of the tubular member.

In some aspects, the techniques described herein relate to a shunt stent, wherein the cut-away window or pattern extends only partially around a circumference of the tubular member to define a preferred bending direction.

In some aspects, the techniques described herein relate to a shunt stent, wherein a hinge is coupled between cut ends of a filament of the braided tubular structure such that the hinge bridges opposing filament segments.

In some aspects, the techniques described herein relate to a shunt stent, wherein the hinge includes opposing tubular end segments that receive and secure the cut ends of the filament.

In some aspects, the techniques described herein relate to a shunt stent, wherein the opposing tubular end segments are secured to the filament by welding, brazing, crimping, swaging, adhesive bonding, or combinations thereof.

In some aspects, the techniques described herein relate to a shunt stent, wherein a plurality of hinges are circumferentially distributed around the enlarged end portion of the braided tubular structure.

In some aspects, the techniques described herein relate to a shunt stent, wherein the hinges are positioned at or near braid crossover locations associated with end-loop curvature.

In some aspects, the techniques described herein relate to a shunt stent, wherein hinges are provided on both proximal and distal enlarged end portions of the shunt stent.

In some aspects, the techniques described herein relate to a shunt stent, wherein the hinges permit the enlarged end portion to articulate to a more acute angle during crimping than would occur in the absence of the hinges.

In some aspects, the techniques described herein relate to a shunt stent, wherein the hinges reduce formation of uncontrolled kinks in the braided tubular structure during radial constraining.

In some aspects, the techniques described herein relate to a shunt stent, wherein the hinges elastically recover upon deployment to restore a predefined expanded geometry of the enlarged end portion.

In some aspects, the techniques described herein relate to a shunt stent, wherein at least one hinge is formed by a localized reduction in cross-section of a filament of the braided tubular structure.

In some aspects, the techniques described herein relate to a shunt stent, wherein the localized reduction in cross-section is formed by grinding, etching, laser ablation, electropolishing, or coining.

In some aspects, the techniques described herein relate to a shunt stent, wherein at least one hinge includes a non-tubular hinge element coupled between filament segments, the hinge element including a leaf-spring, open-frame bridge, or micro-coil structure.

In some aspects, the techniques described herein relate to a shunt stent, wherein the shunt stent further includes one or more braid-locking mechanisms configured to stabilize a deployed diameter of the shunt stent, and wherein the hinges are positioned to avoid interference with the braid-locking mechanisms.

In some aspects, the techniques described herein relate to a shunt stent, wherein the hinges are configured to operate in combination with a delivery system that radially constrains the shunt stent during delivery and releases the shunt stent for self-expansion at a target location.

### BRIEF DESCRIPTION OF THE DRAWINGS

The following figures are included to illustrate certain example aspects of the present disclosure and should not be viewed as exclusive or limiting. The subject matter disclosed is capable of considerable modifications, alterations, combinations, and equivalents in form and function, as will occur to one having ordinary skill in the art and having the benefit of this disclosure. The present disclosure references the drawings as follows:

FIG. 1 illustrates an internal view of a human heart showing a left atrium and associated cardiac anatomy relevant to creation of a shunt.

FIG. 2 illustrates a magnified view of a left atrium, including a left atrial appendage (LAA) and pulmonary veins, illustrating an anatomical relationship suitable for creation of a shunt between the LAA and a pulmonary vein.

FIG. 3A illustrates a side view of an inner shaft of an example shunt creation system.

FIG. 3B illustrates a side view of an outer shaft configured to be positioned over the inner shaft of FIG. 3A.

FIG. 3C illustrates a side view of an outer sheath through which the inner shaft and outer shaft may be advanced.

FIG. 3D illustrates a guidewire usable with the shunt creation system.

FIG. 3E illustrates a support instrument configured to support or position tissue during shunt creation.

FIG. 4 illustrates advancement of a delivery system through a wall of a left atrial appendage and a wall of a pulmonary vein.

FIG. 5 illustrates deployment of a shunt stent between a left atrial appendage and a pulmonary vein using the delivery system.

FIG. 6 illustrates blood flow through a completed shunt between a pulmonary vein and a left atrial appendage.

FIG. 7 illustrates an example technique for creating a shunt between a left atrial appendage and a pulmonary vein without leaving a permanent implant, including direct tissue bonding of apposed walls.

FIG. 8A illustrates a side view of an example clip configured to maintain apposition between a left atrial appendage wall and a pulmonary vein wall.

FIG. 8B illustrates a view of the clip of FIG. 8A rotated approximately ninety degrees.

FIG. 9 illustrates the clip of FIG. 8A deployed to maintain apposition between a left atrial appendage and a pulmonary vein.

FIG. 10 illustrates a perspective view of an example hybrid shunt stent including a strut portion and braided tissue-engaging portions.

FIG. 11 illustrates an end view of the shunt stent of FIG. 10 showing a central shunt lumen.

FIG. 12 illustrates a side view of the shunt stent of FIG. 10 in an expanded, rivet-like configuration.

FIG. 13 illustrates a magnified view of an interface between a strut portion and a braided portion of the shunt stent of FIG. 10.

FIG. 14 illustrates a perspective view of a strut portion of a shunt stent.

FIG. 15 illustrates a side view of the strut portion of FIG. 14.

FIG. 16 illustrates an end view of the strut portion of FIG. 14.

FIG. 17 illustrates a perspective view of a braided tissue-engaging portion of a shunt stent.

FIG. 18 illustrates an end view of the braided tissue-engaging portion of FIG. 17.

FIG. 19 illustrates a side view of the braided tissue-engaging portion of FIG. 17.

FIG. 20 illustrates an example shunt stent formed from a single laser-cut tubular member having free-ended struts configured to be formed into tissue-engaging portions.

FIG. 21 illustrates a perspective view of an example fully braided shunt stent.

FIG. 22 illustrates an end view of the fully braided shunt stent of FIG. 21.

FIG. 23 illustrates a side view of the fully braided shunt stent of FIG. 21.

FIG. 24 illustrates a braided shunt stent including an example locking mechanism positioned at a braid crossover.

FIG. 25A illustrates a braided shunt stent prior to engagement of a locking mechanism during expansion.

FIG. 25B illustrates partial engagement of the locking mechanism of FIG. 25A during expansion.

FIG. 25C illustrates full engagement of the locking mechanism at a target expanded diameter.

FIG. 26A illustrates a dual-sleeve locking mechanism prior to engagement.

FIG. 26B illustrates partial engagement of the dual-sleeve locking mechanism of FIG. 26A.

FIG. 26C illustrates full interlocking engagement of the dual-sleeve locking mechanism.

FIG. 27 illustrates a braided shunt stent including discrete hinge structures positioned along an enlarged end portion.

FIG. 28A illustrates deformation of hinge structures during radial constraining of a braided shunt stent.

FIG. 28B illustrates further articulation of the hinge structures during crimping.

FIG. 28C illustrates elastic recovery of the hinge structures upon deployment of the braided shunt stent.

### DETAILED DESCRIPTION

It will be appreciated by persons skilled in the art that the present disclosure is not limited to what has been particularly shown and described herein. A variety of modifications and variations are possible in view of the teachings herein without departing their scope, spirit, or intent.

While different examples may be described in this specification, it is specifically contemplated that any of the features from the different examples can be used and brought together in any combination. In other words, the features of different examples can be mixed and matched with each other. Hence, while every permutation of features from different examples may not be explicitly shown or described, it is the intention of this disclosure to cover any such combinations, especially as may be appreciated by one of skill in the art.

The terminology used in this disclosure should be interpreted in a permissive manner and is not intended to be limiting. In the drawings, like numbers refer to like elements. Unless otherwise noted, all of the accompanying drawings are not to scale. Unless otherwise noted, the term “about” is defined to mean plus-or-minus 5% of a stated value.

The terms distal or distally generally refer to a direction or area towards an end of a device within a patient (e.g., away from a physician/clinician), while the terms proximal or proximally refer to a direction or area toward an end of a device that remains outside of a patient (e.g., toward or closer to a physician/clinician or handle/hub of a device).

Numerical ranges discussed in this specification should be interpreted as both inclusive numerical ranges and as covering/disclosing a plurality of numbers within the ranges. Specifically, a range should be considered to recite numbers that increment by two decimal places (hundredths) for the purposes of support in the claims (e.g., 0.01, 0.02, 0.03, etc.). Any of these incremented numbers from a range should be understood to have significance and importance in the context of the present specification.

The present specification is generally directed to methods, systems, and devices for creating a shunt between two body lumens. More particularly, the methods, systems, and devices of this specification are especially helpful for creating a shunt between a left atrial appendage (LAA) and a pulmonary vein. Since the pulmonary veins return blood flow to the left atrium of a heart, creating a shunt with the LAA may result in some blood flow to pass from one of the pulmonary veins, through the LAA, and into the left atrium, as well as bidirectionally back. This may help prevent blood circulation from pooling, stagnating, or coagulating from a lack of flow, especially with conditions such as atrial fibrillation that result in abnormal blood flow through the heart.

In some circumstances, creating the aforementioned shunt may have benefits over traditional LAA occlusion treatments. For example, LAA occlusion typically involves implanting an occlusion device within a LAA and allowing it to heal. It is possible that the device does not fully occlude the LAA, which may be difficult to determine during the procedure and hence its effectiveness for reducing stroke risk may be uncertain. In some cases, follow up transesophageal echocardiograms are required at 6 months post-procedure or longer. Even when full occlusion of the LAA occurs, thrombus formation on the device itself remains a risk and therefore the risk of stroke may only be partially reduced by the occlusion procedure.

In contrast, creating a shunt between a pulmonary vein and the LAA may be easier and more reliably achieved. Additionally, the success of the shunt procedure may be better tested, such as with the contrast methods described later in this specification to understand blood flow through the LAA. Additionally, the shunt may be created with a stent device with relatively little exposure to the blood as compared with an occlusion device, especially once healed over, further reducing thrombus creation from the treatment device itself. In some examples, the shunt may be created without any implant device (e.g., via thermal tissue fusion devices).

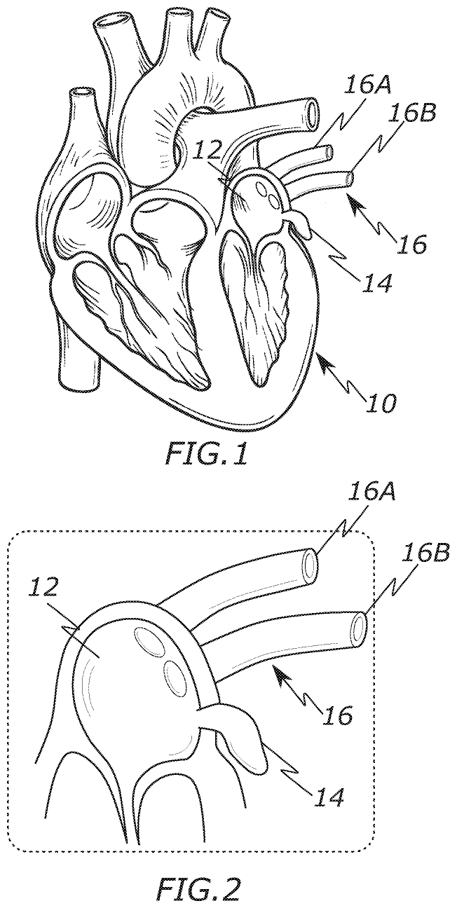

By way of background, FIG. 1 illustrates an internal view of a heart 10. FIG. 2 illustrates a magnified view of a left atrium 12 of the heart 10. A superior pulmonary vein 16A and an inferior pulmonary vein 16B connect to the left atrium 12 to supply oxygen-rich blood. The left atrial appendage, referred to hereafter as the LAA 14 is illustrated as a sack-like structure with an internal space open to the left atrium 12. It should be noted that the view of FIG. 2 and other figures in this disclosure illustrate the LAA 14 as being closest to the inferior pulmonary vein 16B for simplicity of illustration. However, a more accurate three-dimensional illustration may better illustrate the LAA 14 as being closer to the superior pulmonary vein 16A, which is the case in most but not all patients. Generally, the superior pulmonary vein 16A and inferior pulmonary vein 16B will be referred to more generally as a pulmonary vein 16 in connection with creating a shunt with the LAA 14. However, it should be understood that such a shunt may be created with either the superior pulmonary vein 16A or inferior pulmonary vein 16B, depending on the patient's specific cardiac anatomy and other considerations.

Generally, a shunt between the LAA 14 and pulmonary vein 16 may be created with a variety of systems and techniques used for creating shunts at other locations, including catheter/sheath combinations and stent/anastomosis devices for creating the shunt passage. However, the present specification provides some examples of devices, as well as variations thereof, that may be particularly helpful for creating a shunt in such a location. Some of these systems/devices may alternatively be used to create shunts in other locations as well. Hence, while the systems/devices of this specification are primarily described and intended for creating a shunt between a LAA 14 and pulmonary vein 16, other shunt locations are specifically contemplated.

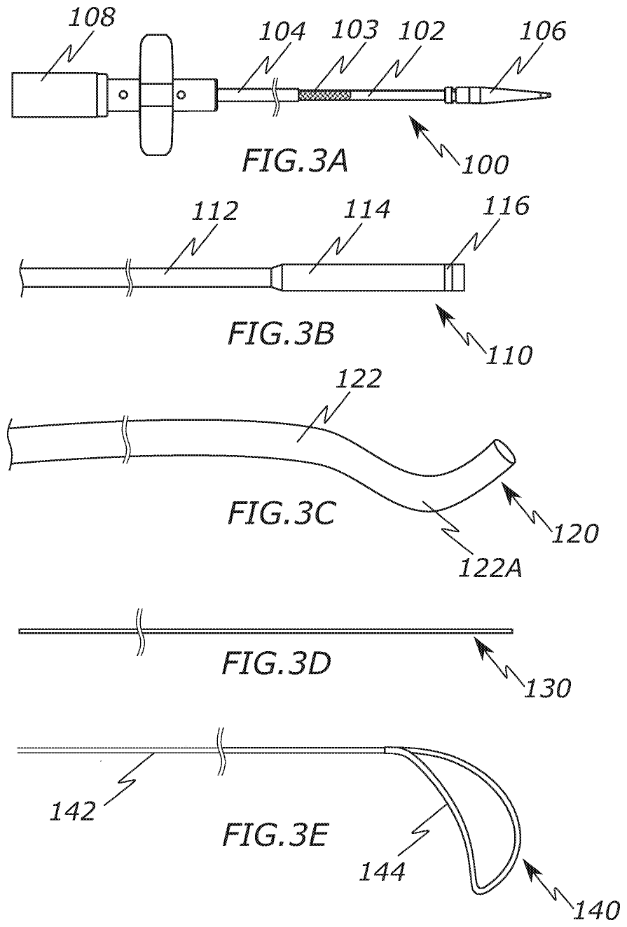

FIGS. 3A, 3B, 3C, 3D, and 3E illustrate side views of some example components of a shunt creation system that may be used to create a shunt between a LAA 14 and pulmonary vein 16. FIG. 3A illustrates an inner shaft 100 on which a shunt stent 150 is crimped, FIG. 3B illustrates an outer shaft 110 that is positioned over the inner shaft 100 and longitudinally slides to maintain or release the shunt stent 150 from the inner shaft 100, FIG. 3C illustrates an outer sheath 120 through which the inner shaft 100 and outer shaft 110 pass through; FIG. 3D illustrates a guidewire 130 that the inner shaft 100 may be advanced over; and FIG. 3E illustrates a support instrument 140 that may be used to position or support the LAA 14.

Turning first to the inner shaft 100 and outer shaft 110, these components may be generally referred to as a stent delivery system since they may be used to deploy the shunt stent 150 during a procedure. The inner shaft 100 may include an inner tube 102 that forms a guidewire passage that opens distally at a distal tip 106 and proximally at a hub 108. This may allow the inner shaft 100 to be advanced over a guidewire 130 that has been advanced to a desired location within a heart 10.

An outer tubular member may be positioned over the inner tube 102 and may extend from a proximal portion (e.g., near or within the hub 108) and to a distal area proximal of the distal tip 106, thereby creating a longitudinal gap or reduced diameter area in which the shunt stent 150 may be crimped. In some examples, a sleeve 103 may be positioned on the inner tube 102 and within the reduced diameter area to help retain the shunt stent 150 as it is deployed.

In some examples, the outer shaft 110 may generally be a tubular shape with a lumen sized to fit over the inner shaft 100. The outer shaft 110 may have a relatively larger diameter distal region 114 (both externally and internally with the lumen) and a relatively smaller diameter proximal region 112. The relatively smaller diameter proximal region 112 extends most of the length of the outer shaft 110 while the relatively larger diameter distal region 114 may have a length of about the length of the reduced diameter region of the inner shaft 100 (e.g., the same size or somewhat larger in length). This may allow for the relatively smaller diameter proximal region 112 to provide extra room with the lumen of the outer sheath 120 and thereby provide more maneuverability and simultaneous access with other tools. This may also allow an additional “stationary layer” to exist in the proximal region 112 which isolates motion of the outer shaft 110 from the access site (either skin or introducer sheath). In other words, it allows the motion of the inner layers to not be relative to the access site. Alternatively, the 110 may have a uniform outer diameter. A radiopaque marker 116 may also be included at a distal end of the 110 for visualization.

The outer sheath 120 may have a generally tubular body 122 with a lumen extending between its proximal and distal end. This allows the outer sheath 120 to be passed over a guidewire 130 and/or the delivery system (e.g., inner shaft 100 and outer shaft 110) to be advanced through to a target location. In some examples, the outer sheath 120 may have a curve 122A near its distal end, such as within an inclusive range of about 30 degrees to about 60 degrees relatively to a longitudinal axis of the outer sheath 120 (e.g., about 45 degrees). This curve 122A may be pre-formed into the generally tubular body 122 so that it occurs when the generally tubular body 122 is unconstrained. This may help angle the distal opening of the inner lumen of the outer sheath 120 in a direction helpful for accessing the LAA 14. Additionally or alternatively, the outer sheath 120 may include a steering mechanism that allows the distal portion of the generally tubular body 122 to be bent or angled relative to proximal portions of the generally tubular body 122 via user accessible controls at a proximal end of the generally tubular body 122.

FIG. 3D illustrates a guidewire 130 that may be sized to pass through the lumen of the inner tube 102 of the inner shaft 100. In some examples, the guidewire 130 may be a relatively standard guidewire as is known in the art. In other examples, the guidewire 130 may be configured to pass through the walls of the LAA 14 and pulmonary vein 16. For example, the guidewire 130 may have a sharpened tip or may be an RF guidewire that pierces tissue with RF energy. In some examples, a standard guidewire may be used for some portions of the procedure while the sharpened or RF guidewire may be swapped out to be used for other portions of the procedure. In other examples, the distal tip 106 of the inner shaft 100 may include an electrode that may be supplied with RF energy to pierce the tissue walls.

FIG. 3E illustrates a support instrument 140 that may be used to support, lift, or otherwise help create apposition between the LAA 14 and the pulmonary vein 16. For example, the support instrument 140 may be advanced or retracted to achieve the desired apposition. The support instrument 140 may generally have an elongated wire shaped body 142 that extends proximally (e.g., to a proximal end of the outer sheath 120) and also includes a distal portion 144 that forms a relatively enlarged shape as compared to the elongated wire shaped body 142. In the present example, the distal portion 144 forms a wire loop shape which may distributed pressure against an interior of the LAA 14 while providing an opening through its center for the inner shaft 100 and outer shaft 110 to pass through to create the shunt passage. However, other shapes of the distal portion 144 are also possible, such as a “V” shape, a “T” shape, a sinusoidal pattern, a three-dimensional mesh shape, or similar variations.

In certain examples, the support instrument includes one or more radiopaque markers positioned on or near the distal tissue-engaging region to fluoroscopically delineate the LAA wall and/or PV wall during shunt creation. Such markers may include platinum-iridium bands, tantalum markers, or radiopaque sleeves integrated into the loop or other distal geometry. Under fluoroscopy, the markers provide a positional reference that facilitates orientation, apposition, and deployment of the shunt-forming instrument.

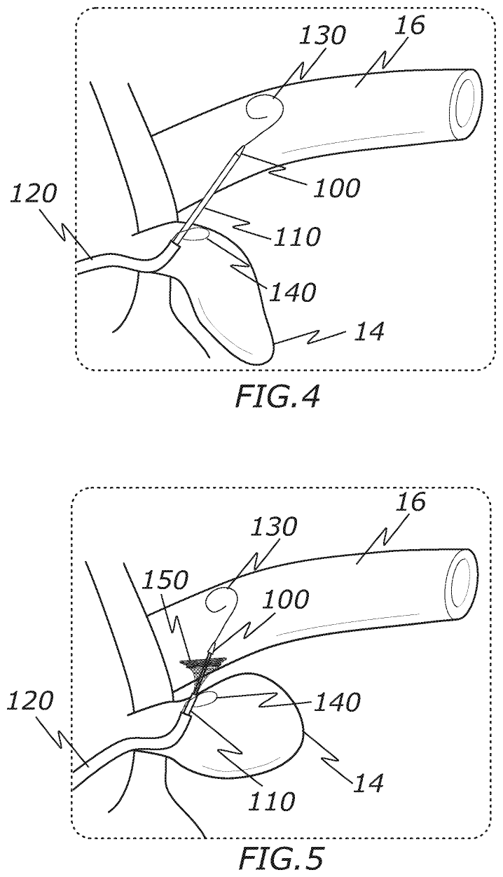

FIGS. 4, 5, and 6 illustrate one example procedure of creating a shunt between a LAA 14 and a pulmonary vein 16 using the aforementioned devices. However, it should be understood that a variety of other techniques currently known by those of skill in the art may be used to achieve the same or similar shunt.

Turning first to FIG. 5, the outer sheath 120 may first be advanced outside of or inside of the LAA 14. In some examples, a guidewire 130 may be first advanced so that its distal end is near or within the LAA 14 and the outer sheath 120 may be advanced over the guidewire 130. If the outer sheath 120 is a steerable sheath, it may be angled as desired so that its opening is directed towards the desired pulmonary vein 16 to create a shunt with (e.g., superior pulmonary vein 16A or inferior pulmonary vein 16B).

In some examples, the support instrument 140 may be advanced distally out of the outer sheath 120 and pressed against a wall of the LAA 14 so that the LAA 14 moves closer to the pulmonary vein 16. This may be performed prior to piercing the walls of the LAA 14 and pulmonary vein 16, at the same time, or after.

Next, the walls of the LAA 14 and pulmonary vein 16 may be pierced or crossed. As previously discussed, this may be achieved in several ways. For example, the guidewire 130 may have a needle point for physically piercing the walls or may have an RF electrode for delivering RF energy to pass through. Alternatively, such guidewires may be swapped out for a standard guidewire that may have been initially used. In another alternative example, the guidewire 130 may be a standard, non-piercing guidewire and the inner shaft 100 may have an RF electrode on its distal tip 106 that is configured to deliver RF energy and allow for crossing the walls of the LAA 14 and pulmonary vein 16.

Next, the inner shaft 100 and outer shaft 110 are advanced through the wall of the LAA 14 and the wall of the pulmonary vein 16, as seen in FIG. 4. As seen in FIG. 5, once the delivery system (e.g., inner shaft 100 and outer shaft 110) is positioned at a desirable position between the two body lumens, the outer shaft 110 may be withdrawn proximally allowing the shunt stent 150 to radially expand. In some examples, the shunt stent 150 may be self-expanding. In some examples, the inner shaft 100 may include an inflatable balloon under the shunt stent 150 that may be inflated to assist or cause expansion. In some examples, the delivery system may be removed and a separate balloon catheter may be positioned within the shunt stent 150 to fully expand the shunt stent 150. In other examples, the shunt stent 150 may have other mechanism to cause expansion, such as pull wires that pull locations on proximal and distal sides of the shunt stent 150 toward each other to longitudinally compress the shunt stent 150.

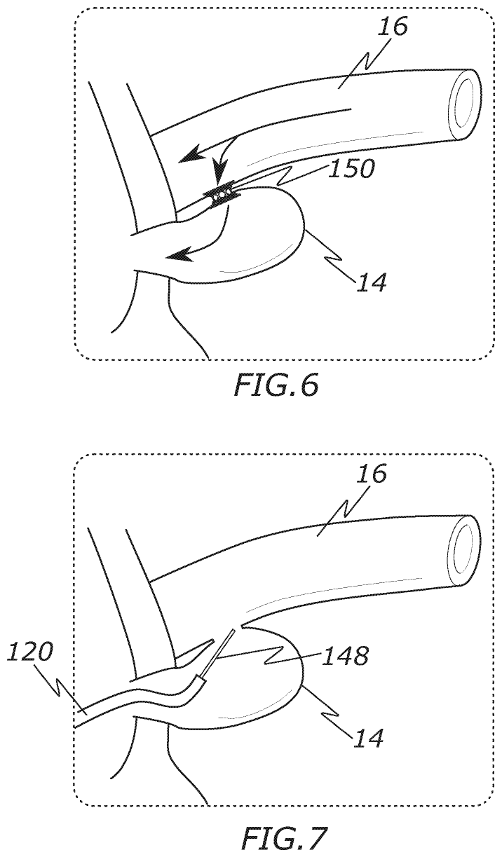

Finally, in FIG. 6, a shunt or shunt passage is created between the LAA 14, through the shunt stent 150, to the pulmonary vein 16. As noted by the arrows, as blood flow passes through the pulmonary vein 16, some is diverted into the shunt created by the shunt stent 150 and further into the LAA 14 and out into the left atrium 12.

At this point, the effectiveness of the newly created shunt may be determined. In one example, an angiography washout retention test may be performed to help determine the effectiveness of the shunt and therefore help predict stroke risk. Angiographic washout testing of the LAA typically evaluates the clearance characteristics of contrast material within the appendage. During conventional LAA angiography, contrast is injected into the LAA cavity, and fluoroscopic imaging is used to observe the rate at which the contrast dissipates from the appendage and re-enters the left atrial circulation. Slow or incomplete washout is generally interpreted as an indicator of reduced LAA mechanical function or impaired flow dynamics; conditions that are associated with blood stasis and an elevated risk of thrombus formation. Washout behavior may be qualitatively or semi-quantitatively assessed by monitoring contrast persistence over multiple cardiac cycles, noting whether the contrast clears rapidly, gradually, or remains retained within the appendage.

In certain examples, washout testing may be performed following the creation of the shunt between the pulmonary vein 16 and the LAA 14. Since the shunt establishes a direct flow pathway intended to introduce pulmonary venous blood into the LAA, it thereby augments circulation within the appendage. After formation of the shunt, contrast material may be introduced into the LAA 14 using standard angiographic techniques. Fluoroscopic imaging is then used to evaluate the washout characteristics of the contrast in the presence of the newly created flow pathway. The rate and pattern of contrast clearance may be compared to pre-shunt baselines or to expected physiologic norms to determine the effectiveness of the shunt in promoting flow through the LAA 14.

In some implementations, the washout assessment includes measuring the number of cardiac cycles required for substantial or complete clearance of contrast from the LAA 14, identifying regions of persistent contrast retention, and/or evaluating directional flow patterns induced by the shunt. Enhanced washout, such as a reduction in contrast persistence or the elimination of stagnant zones, may indicate that the shunt is effectively increasing blood turnover within the appendage. Conversely, persistent retention or minimal change in washout behavior may suggest inadequate shunt function, suboptimal positioning, or insufficient flow communication between the pulmonary vein and the LAA 14. The washout test therefore provides a functional evaluation method for determining whether the shunt achieves its intended purpose of improving LAA flow dynamics, which may be relevant to reducing stasis-related stroke risk.

In certain examples, a machine-learning model may be developed to automatically evaluate contrast washout characteristics of the LAA 14 following creation of a shunt between the pulmonary vein 16 and the LAA 14. A training dataset may be assembled from fluoroscopic image sequences obtained from multiple subjects who have undergone the shunt procedure. Each dataset may include time-resolved angiographic images capturing the injection of contrast into the LAA 14 and the subsequent washout over several cardiac cycles or elapsed time. The datasets may be annotated with one or more reference indicators, such as qualitative or semi-quantitative assessments of washout speed, contrast persistence, directional flow patterns, or other physiologic markers of LAA 14 circulation. These annotations may be generated by expert reviewers or by computational extraction of predefined image-based metrics.

The training process may include preprocessing steps such as temporal normalization, segmentation of the LAA region, motion compensation, and extraction of pixel-intensity time curves representing contrast clearance. The machine-learning model (e.g., a convolutional neural network, recurrent neural network, transformer-based architecture, or hybrid model) may be trained to identify patterns within the fluoroscopic sequences that correlate with the annotated washout characteristics. During training, the model may learn to associate specific temporal and spatial contrast-dissipation features with corresponding levels of shunt-induced flow augmentation. Additionally or alternately, the model may also process patient stroke data associated with each corresponding patient to associate the shunt flow augmentation and similar patterns with later stroke risk. The model may additionally or alternatively process existing data on LAA 14 flow characteristics and stroke risk to help correlate different shunt/LAA flow patterns with stroke risk. The resulting trained model may therefore encode a mapping between angiographic washout behavior and the functional performance of the shunt, as well as stroke risk.

Once trained, the model may be applied to fluoroscopic imaging obtained from a new patient following creation of the pulmonary-vein-to-LAA shunt. The system may receive the patient's angiographic contrast washout sequence, perform automated preprocessing, and generate an output representing the predicted washout characteristics or an assessment of shunt effectiveness. In some implementations, the model may output a quantitative score, a categorical classification, or a visualization highlighting regions of delayed washout or improved flow. The predicted values may be compared to baseline or population-derived reference ranges to determine whether the newly created shunt is producing the intended enhancement of LAA circulation and what the risk of stroke may be.

In certain examples, the system may further incorporate adaptive learning, enabling the model to be periodically updated as additional patient data becomes available. This allows the model to refine its predictive accuracy over time and maintain performance across variations in anatomy, shunt configuration, imaging parameters, and procedural techniques. The Al-based evaluation method therefore provides a computational tool for assessing the functional impact of the shunt on LAA washout dynamics using fluoroscopic imaging data.

In addition to fluoroscopy, any suitable medical imaging modality may be used to acquire time-resolved image sequences for evaluating blood washout from the LAA. For example, ultrasound-based imaging, such as intracardiac echocardiography (ICE) or transesophageal echocardiography (TEE), may be used to visualize blood flow, dispersion, or microbubble washout patterns within the LAA. Magnetic resonance imaging (MRI) techniques, including real-time or time-resolved contrast-enhanced MRI, may be used to characterize contrast clearance, flow vectors, or perfusion patterns following creation of the shunt. Computed tomography (CT), including dynamic or multiphase CT angiography, may similarly depict temporal changes in contrast distribution within the LAA and thereby enable quantitative or semi-quantitative assessment of washout. These modalities may be used individually or in combination, and images obtained from any of them may serve as inputs for determining washout metrics, directional flow patterns, or other indicators of shunt effectiveness as described herein.

FIG. 7 illustrates another example technique of creating a shunt between the LAA 14 and pulmonary vein 16 without leaving a permanent shunt stent or other implant device within the shunt passage. Instead, after an opening is formed through the adjacent walls of the LAA 14 and pulmonary vein 16, the opposing tissue surfaces may be directly bonded to one another to create and maintain a flow pathway. Such approaches may leverage existing tissue-apposition, tissue-fusion, or tissue-bonding technologies that are already known for creating anastomoses or sealed passages between adjacent body lumens.

In these examples, once apposition between the LAA wall and the pulmonary vein wall is achieved, an opening may be created through both walls using any of the crossing techniques described elsewhere in this specification, such as mechanical puncture, radiofrequency (RF) energy, laser energy, or other energy-assisted penetration methods. Following formation of the opening, the tissue surrounding the opening on each side may be brought into close contact and secured together so that the two luminal walls heal as a unified structure defining the shunt passage. The resulting bonded interface may thereby serve as the structural support for the shunt without the need for a metallic or polymeric stent.

In some examples, direct tissue bonding may be achieved using thermal tissue fusion techniques. For instance, a bonding catheter 148 may use RF energy, resistive heating, ultrasonic energy, or other controlled thermal energy that is applied to the apposed tissue margins surrounding the opening to denature collagen and elastin within the tissue layers, allowing the tissue surfaces to fuse together upon cooling. Such tissue fusion may create a circumferential seal around the shunt passage that resists separation under physiologic pressure while maintaining an open lumen between the pulmonary vein and the LAA. Energy delivery parameters may be selected to promote bonding while limiting collateral thermal injury.

Additionally or alternatively, the shunt may be stabilized using bioadhesives or sealants applied to the interface between the LAA wall and pulmonary vein wall. In such examples, a biocompatible adhesive, such as a fibrin-based adhesive, synthetic polymer adhesive, or other known medical sealant, may be delivered to the tissue interface after the opening is formed. The adhesive may secure the two tissue layers together during the healing period and may optionally be resorbable over time, allowing the bonded tissue to remodel naturally into a stable shunt passage.

In further examples, mechanical tissue-apposition elements may be used transiently to facilitate bonding and healing without remaining as permanent implants. For example, temporary clamps (e.g., as described elsewhere in this application), expandable anchors, compression members, suturing elements, or staple-like structures may be deployed to hold the LAA wall and pulmonary vein wall in apposition while tissue fusion, adhesive bonding, or natural healing occurs. These elements may be configured to be removed after a desired bonding period or to resorb over time, leaving behind a shunt formed primarily by healed native tissue.

In stentless examples, the resulting shunt passage may have reduced exposure of foreign material to circulating blood as compared to a stent-based shunt, particularly after healing is complete. This may reduce thrombogenic surface area and may allow the shunt geometry to conform more naturally to patient-specific anatomy. As with stent-based examples, the effectiveness of the stentless shunt may be evaluated using contrast washout testing, flow measurements, or other functional assessments described elsewhere in this specification to confirm improved circulation through the LAA.

In examples with a shunt stent 150 or with the creation of a stentless shunt, it may be helpful to use a clamp or anchor that helps move the LAA 14 and/or pulmonary vein 16 into apposition against each other. Hence, the present specification includes methods and devices for clamping/anchoring the LAA 14 and pulmonary vein 16 against each other prior to, during, and/or after creation of a shunt.

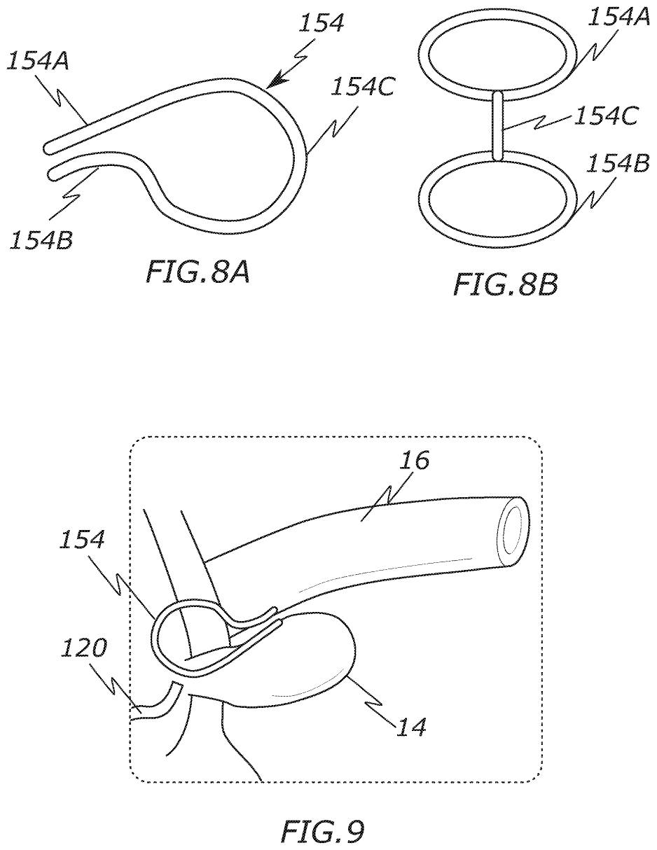

One example can be seen in FIG. 8A which illustrates a side profile view of a clamp 154 that is configured to draw or move the LAA 14 against the pulmonary vein 16 and maintain apposition. FIG. 8B illustrates a view of the clamp 154 at about 90 degrees to that of FIG. 8A. FIG. 9 illustrates a side view of the clamp 154 clamping the LAA 14 against the pulmonary vein 16.

In some examples, the clamp 154 may extend from within the pulmonary vein 16, into the left atrium 12, and into the LAA 14 so that opposing contact end regions 154A, 154B at distal and proximal ends engage tissue on the venous and appendage sides. A curved middle portion 154C may span between these end regions 154A, 154B to conform to or navigate through the native atrial geometry and to distribute contact forces along the interposed tissue. Such a clamp 154 can support stent-based or stentless shunt creation approaches, including the direct tissue-bonding methods described elsewhere in this specification.

In one example, the clamp 154 includes a generally arcuate middle portion 154C configured to follow a path from a first end region 154A positioned within the pulmonary vein 16, through the left atrium 12, to a second enlarged tissue-engaging area end region 154B positioned within the LAA. The enlarged end regions may be formed as loops (e.g., closed wire loops or loop-like frames, as seen in FIG. 8B) or as functionally similar shapes (e.g., teardrops, D-shapes, rounded paddles, open loops, Y-shapes, or multi-filament petals) that present an atraumatic, broad bearing surface to minimize localized pressure while providing positional stability. The curved middle portion may have a resting radius sized to bias the LAA wall and pulmonary vein wall toward one another when the enlarged areas are seated, thereby promoting tissue apposition suitable for shunt formation and bonding. By way of example, the curved middle portion may have a resting radius within an inclusive range of about 5 mm to about 25 mm, such as about 8-15 mm, sized to bias the LAA wall and pulmonary vein wall toward one another during seating.

The clamp body may be formed from an elastic or superelastic member (e.g., a nitinol wire or laser-cut struts) that permits reversible deformation for delivery through a catheter and recovery to an expanded clamping configuration upon deployment. In some examples, the wire gauge, heat-set curvature, and loop diameters are selected so that the clamp provides a contact force sufficient to maintain wall-to-wall apposition while allowing pulsatile compliance and limiting peak interface pressure. Radiopaque markers may be positioned at or near the loop apices and/or along the middle segment to facilitate fluoroscopic positioning. Surface features such as rounded edges, soft sleeves, or low-friction coatings may further reduce trauma to the endothelial surfaces during placement and retrieval.

The clamp 154 may be sized and configured for transcatheter delivery. In one example, the clamp is constrained within a delivery catheter in a low-profile, elongated form with the end regions 154A, 154B partially straightened and the curved middle portion 154C reduced in arc length (straightened or further bent). The catheter may be advanced into the left atrium using conventional transseptal techniques. Under imaging guidance, a distal portion of the clamp may first be advanced into the pulmonary vein where the first enlarged area expands to engage the venous wall. The catheter may then be retracted to arch the curved middle portion 154C through the left atrium 12, and the proximal enlarged area may be advanced into the LAA where it expands to seat against the interior appendage wall. Once both end regions 154A, 154B are seated, the curved middle segment biases the two walls into contact to facilitate crossing and/or bonding. After the shunt has been created and stabilized (e.g., following tissue fusion or adhesive cure), the clamp may be collapsed and withdrawn through the catheter, or in some examples configured to resorb over time.

Various optional features may be incorporated. For example, one or both end regions 154A, 154B may include an open-center loop that leaves a working aperture through which needles, guidewires, RF crossing elements, or bonding catheters can be passed to create and stabilize the shunt tract while the clamp maintains apposition. The loop shapes may include localized flex joints or torsion-relief turns to accommodate respiratory and cardiac motion without losing seating. In some examples, the curved middle portion 154C may include a gentle torsional pre-bias to align the clamp plane with the anatomical plane connecting the pulmonary vein 16 and the LAA 14. The clamp may further include temporary locking or ratcheting features, tethering filaments for retrieval, and atraumatic end caps. When used with stentless, direct tissue-bonding approaches, the clamp can serve as a temporary external scaffold that maintains consistent wall contact throughout the bonding cycle and early healing period.

The curved middle portion 154C may have a generally uniform, symmetrical curvature or may have an asymmetrical curvature between the end regions 154A, 154B, as seen in FIG. 8A. While the end regions 154A, 154B are shown as generally planar, they may also be curved. For example, the proximal and distal ends of the end regions 154A, 154B may be angled away from each other so that the middle of each of the end regions 154A, 154B are closest to each other.

Materials, finishes, and geometries disclosed for other support instruments in this specification may be applied to the clamp. By way of example, wire-loop distal structures described for positioning within the LAA 14 can be adapted to form the proximal enlarged area of the clamp, while the venous side loop may be contoured to the elliptical profile of a pulmonary vein ostium. The clamp may be provided in a set of sizes to accommodate patient-specific anatomy, and the delivery system may include exchangeable introducers or sheaths with steerable tips to assist in orienting the clamp along the desired apposition line.

In an alternative example, the clamp 154 may be deployed without traversing the left atrium by first creating a puncture or crossing directly through the adjacent walls of the LAA 14 and the pulmonary vein 16. After the opening is formed, the clamp 154 may be advanced through the puncture such that the curved middle portion 154C is positioned within the shunt tract extending between the two lumens, rather than spanning through the atrial chamber. In this configuration, the end regions 154A, 154B on opposite ends of the clamp 154 may expand on opposing sides of the tissue interface, with one enlarged area engaging tissue on the pulmonary vein side and the other enlarged area engaging tissue on the LAA side, thereby drawing the walls together around the opening. This arrangement may maintain localized apposition of the tissue margins surrounding the puncture to facilitate shunt stabilization, tissue bonding, or healing, while potentially reducing interaction of the clamp with the broader left atrial anatomy.

In one example method, a delivery catheter containing the clamp 154 in a constrained configuration is advanced to the left atrium 12 using a transseptal approach. The end region 154B is then deployed from the catheter and positioned within the pulmonary vein 16 so that it expands to engage the venous wall. The catheter is subsequently retracted to allow the curved middle portion 154C of the clamp 154 to extend across the intervening anatomy, and the proximal end region 154A is advanced into the LAA 14 where it expands to engage appendage tissue. The opposite sequence of deploying within the LAA 14 first and subsequently in the pulmonary vein 16 is also possible. With both enlarged areas seated, the curved middle portion biases the LAA 14 and pulmonary vein 16 walls toward each other to maintain apposition for shunt formation, tissue bonding, or healing, after which the clamp may be withdrawn or otherwise removed as desired.

Due to the unique position and geometry of the LAA 14 and pulmonary vein 16, certain features and designs of a shunt stent 150 may be helpful for successfully creating a shunt therebetween.

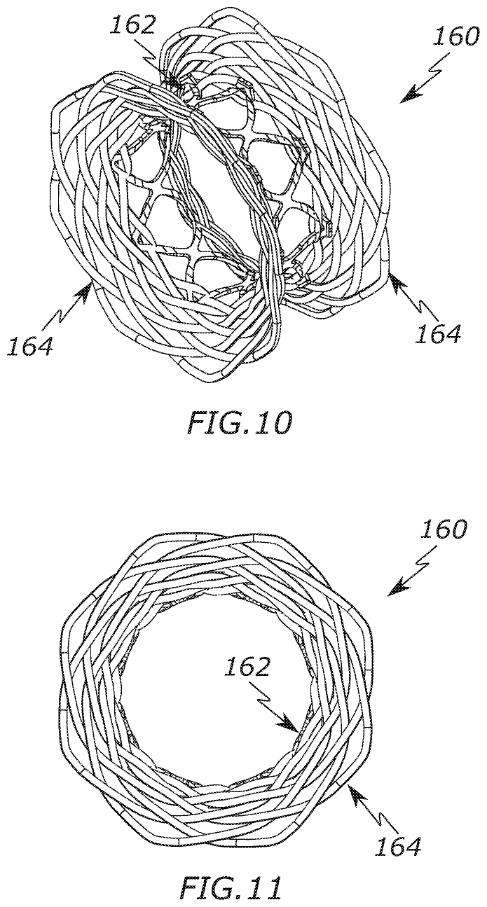

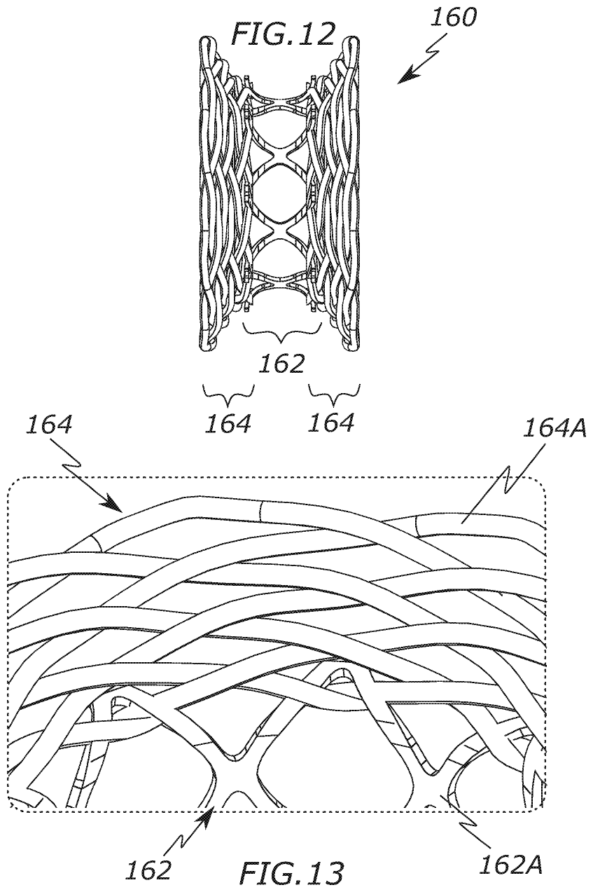

FIGS. 10-13 illustrate one example of a shunt stent 160 that includes both a strut portion 162 and a woven or braided portion 164. Generally, the strut portion 162 may form a middle region of the one example shunt stent 160 to help create a reinforced shunt passage of a desired diameter while the braided portion 164 may help radially expand and longitudinally retract towards each other to help “clamp” or seal the tissue around the strut portion 162. FIG. 10 illustrates a perspective view of the shunt stent 160, FIG. 11 illustrates an end view of the shunt stent 160 illustrating the passage therethrough, FIG. 12 illustrates a side view of the shunt stent 160 showing its radially expanded state and “rivet” shape, and FIG. 13 illustrates a magnified view of the interface between the strut portion 162 and the woven or braided portion 164.

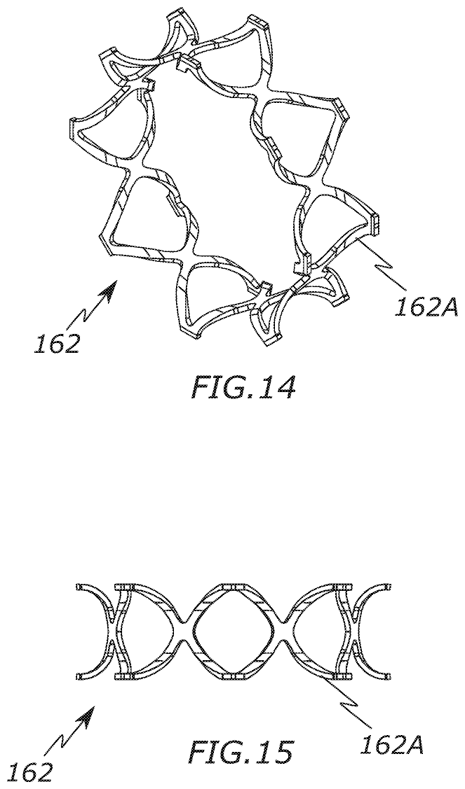

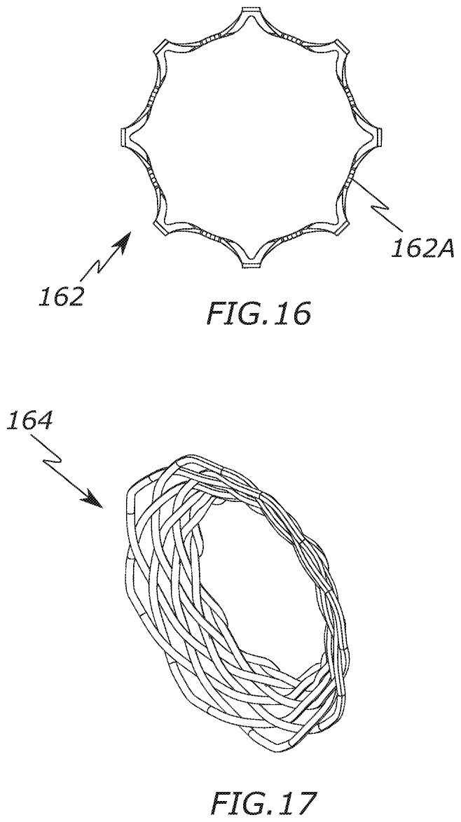

The strut portion 162 may include a plurality of struts 162A arranged to form a repeating cell pattern along a longitudinal axis of the shunt stent 160. The strut portion 162 may be seen alone in FIG. 14 (perspective view), FIG. 15 (side view), and FIG. 16, (end view). In some examples, the struts 162A are formed from a laser-cut tubular member, such as a metallic tube, and may define cells having a variety of shapes, including diamond-shaped, rhomboid, hexagonal, circular, or other known stent cell geometries. In the present example, only a single row of cells are formed by the struts 162A, however, several proximal-to-distal rows of cells are also possible. The strut geometry, thickness, and cell size may be selected to achieve a desired balance between radial strength, flexibility, and foreshortening behavior during expansion. In some examples, the strut portion 162 is plastically deformable and radially self-expanding, while other examples include expansion by balloon catheter.

As seen best in FIG. 15, the strut portion 162 may have a radially expanded shape that curves radially outward relative to longitudinally middle/inner portions. This may help the braided portions 164 to expand radially outward from the strut portion 162 to achieve a “rivet” shape. In some examples, the proximal and distal ends of the strut portion 162 curve to about 90 degrees relative to their longitudinally middle regions.

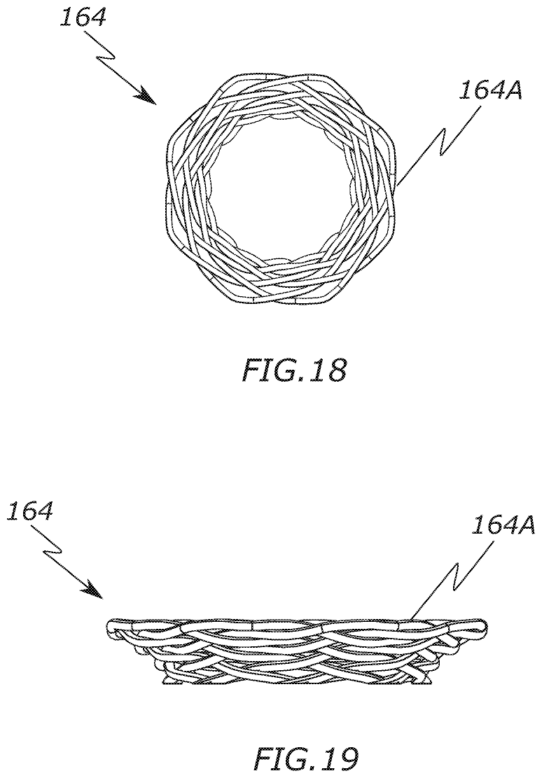

The braided portion 164 is also shown alone in FIG. 17 (perspective view), FIG. 18 (end view), and FIG. 19 (side view). Each braided portion 164 may include one or a plurality of elongate filaments or wires 164A that are braided, woven, or otherwise interlaced to form a tubular braid extending from each proximal/distal end of the strut portion 162. The braided portion 164 may be formed from metallic wires (e.g., nitinol, stainless steel, cobalt-chromium alloys), polymeric filaments, or combinations thereof. In some examples, the braided portion 164 is shape-set such that, upon release from a constrained delivery configuration, it radially expands and longitudinally foreshortens relative to its constrained state, in some examples forming concave shapes. This expansion and foreshortening may cause the braided portions 164 on opposing sides of the strut portion 162 to draw tissue toward the central region of the stent, thereby creating a rivet-like or clamping effect that captures and compresses tissue between the braided portions.

Each braided portion 164 may be coupled to longitudinally proximal or distal ends of the strut portion 162 in a variety of ways. In some examples, the wires 164A forming the braided portion 164 are braided directly through openings or cells formed by the struts 162A of the strut portion 162, thereby mechanically interlocking the braided and strut components. In other examples, the braided portion 164 may be attached to the strut portion 162 by welding, brazing, soldering, crimping, adhesive bonding, or combinations thereof. In further examples, separate attachment components such as collars, sleeves, rings, or eyelets may be used to secure the braided portion 164 to the strut portion 162. These attachment techniques may be employed individually or in combination to provide a robust mechanical connection while accommodating relative deformation during deployment.

The strut portion 162 may have a relatively higher strength and more consistent expanded diameter than if the entire shunt stent 160 was composed of braided wires. Additionally, the braided portion 164 of each proximal/distal side may better conform to the tissue walls of the LAA 14 and pulmonary vein 16 than if the entire shunt stent 160 was composed of laser cut struts. Hence, this design may incorporate some of the best features of both constructions.

In some examples, the shunt stent 160 (or any other shunt stent variation described in this specification) may include a semi-porous or non-porous layer on its outside, on its inside, or on both outside and inside. This layer or layers may help create a seal for the shunt passage created by the shunt stent 160. Such coverings may include ePTFE, polyurethane, silicone, electro spun materials; textile material, a textile material with a polymeric coating or treatment, or similar variations. The layer or layers may cover only the strut portion 162, only the braided portion 164, or both portions. Further, both portions may be covered with different types of material that better accommodate their radial expansion.

In use, the shunt stent 160 may be delivered to the target location in a radially compressed configuration within a delivery system (e.g., the inner shaft 100 and outer shaft 110). Upon deployment, the strut portion 162 expands to establish a primary shunt lumen between the body lumens, while the braided portions 164 expands radially outwardly and foreshorten to engage tissue on opposing sides of the shunt passage. This configuration may help retain the stent 160 in position, promote sealing around the shunt tract, and reduce the likelihood of leakage or migration. While shown as including braided portions 164 on both ends of the strut portion 162, other arrangements are also contemplated, including examples with a braided portion on only one side or with multiple braided sections distributed along the length of the stent.

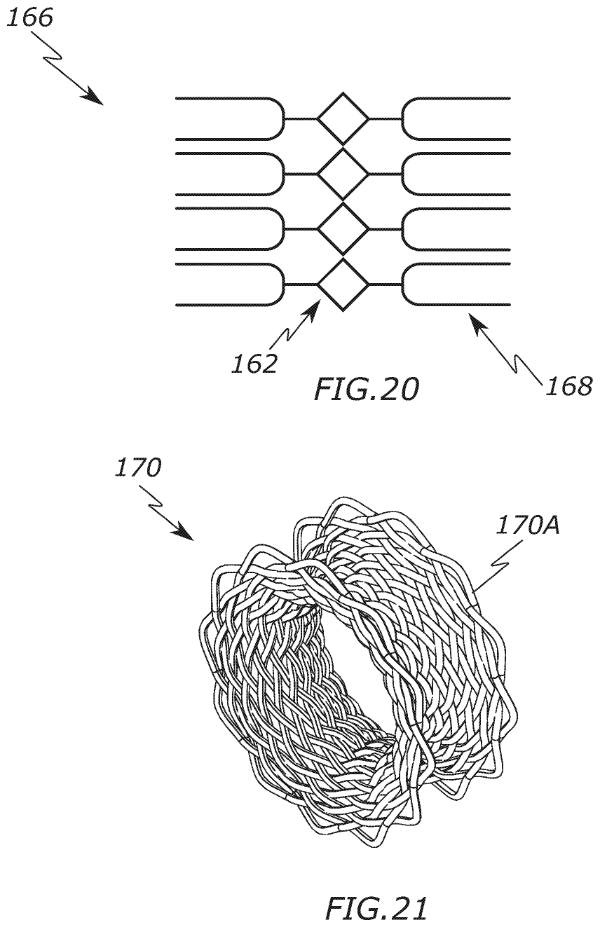

FIG. 20 illustrates another example of a shunt stent 166 (shown in a flattened, pre-assembled state) that is similar in function to the shunt stent 160, but differs in that both the strut portion 162 and the tissue-engaging end portions are formed from a single, continuous laser-cut pattern. In this example, the strut portion 162 includes a plurality of struts 162A arranged to form one or more rows of cells, such as diamond-shaped or other known stent cell geometries, which define a central shunt lumen when expanded. Extending from each longitudinal end of the strut portion 162 are multiple struts 168A that each have a free ends and another that is integrally formed with the strut portion 162, rather than being separately fabricated braided components.

As shown in the flattened pattern of FIG. 20, the free-ended struts 168A extending from each end of the strut portion 162 may be subsequently formed, woven, or braided together to create end portions 168 that function similarly to the braided portions 164 described with respect to shunt stent 160. In some examples, these free-ended struts 168A are interlaced with one another and shape-set to radially expand and longitudinally foreshorten upon deployment, thereby forming tissue-engaging regions that create a rivet-like or clamping effect on opposite sides of the shunt passage. Because the strut portion 162 and the end portions 168 are derived from a single laser-cut structure, the resulting shunt stent 166 may provide a continuous load path between the central lumen-defining region and the tissue-engaging regions, while reducing the need for separate attachment steps such as welding or crimping.

In use, the shunt stent 166 may be delivered in a radially constrained configuration and deployed in a manner similar to shunt stent 160. Upon expansion, the strut portion 162 establishes and maintains the diameter of the shunt passage, while the braided or interlaced end regions 168 formed from the free-ended struts 168A expand outwardly and foreshorten to engage tissue on opposing sides of the opening. This configuration may promote secure retention, sealing around the shunt tract, and conformance to surrounding anatomy, while benefiting from the manufacturing simplicity and structural continuity of a single-piece, laser-cut construction.

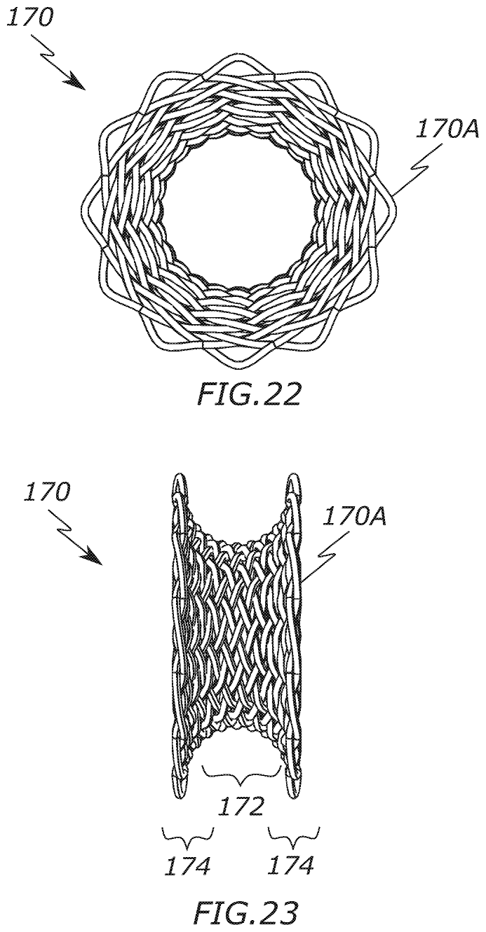

Another example of a fully braided shunt stent 170 may be seen in FIGS. 21-23. FIG. 21 illustrates a perspective view, FIG. 22 illustrates an end view, and FIG. 23 illustrates a side view of the fully braided shunt stent 170. In this example, the shunt stent 170 is composed of a single elongate filament or wire 170A braided with sections of itself, or a plurality of such filaments 170A braided together to form a continuous tubular braid. The fully braided shunt stent 170 may be configured to define a central flow passage and to engage tissue on opposing sides of an opening between adjacent body lumens, such as between a pulmonary vein 16 and a left atrial appendage 14.

In an expanded configuration, the fully braided shunt stent 170 may include a middle portion 172 having a relatively smaller diameter and opposing end portions 174 having relatively larger diameters that form flanges or enlarged tissue-engaging regions. The end portions 174 may be configured to expand radially outward and longitudinally foreshorten relative to the middle portion 172 so as to engage tissue on opposite sides of a shunt tract, thereby providing a rivet-like or clamping effect that resists migration and promotes sealing around the opening. The middle portion 172 may define and maintain the effective diameter of the shunt passage between the body lumens.

The expanded shape of the fully braided shunt stent 170 may be established in a variety of ways. In some examples, the braided structure is formed in a substantially uniform cylindrical configuration and subsequently heat set to define the smaller-diameter middle portion 172 and the larger-diameter end portions 174. In other examples, the expanded geometry may be achieved by varying the braiding pattern, pitch, or braid angle along the longitudinal length of the stent as it is braided, such that the middle portion 172 and end portions 174 inherently assume different diameters upon expansion. These techniques may be used alone or in combination to achieve a desired deployed profile, radial force distribution, and foreshortening behavior.

In use, the fully braided shunt stent 170 may be delivered in a radially constrained configuration within a catheter or sheath and released at the target location. Upon release, the braided structure expands toward its heat-set or braid-defined shape, with the end portions 174 engaging tissue on opposite sides of the shunt passage and the middle portion 172 forming a flow lumen therebetween. The fully braided construction may provide enhanced flexibility and conformability to surrounding anatomy, while reducing manufacturing complexity by eliminating separate strut and attachment components.

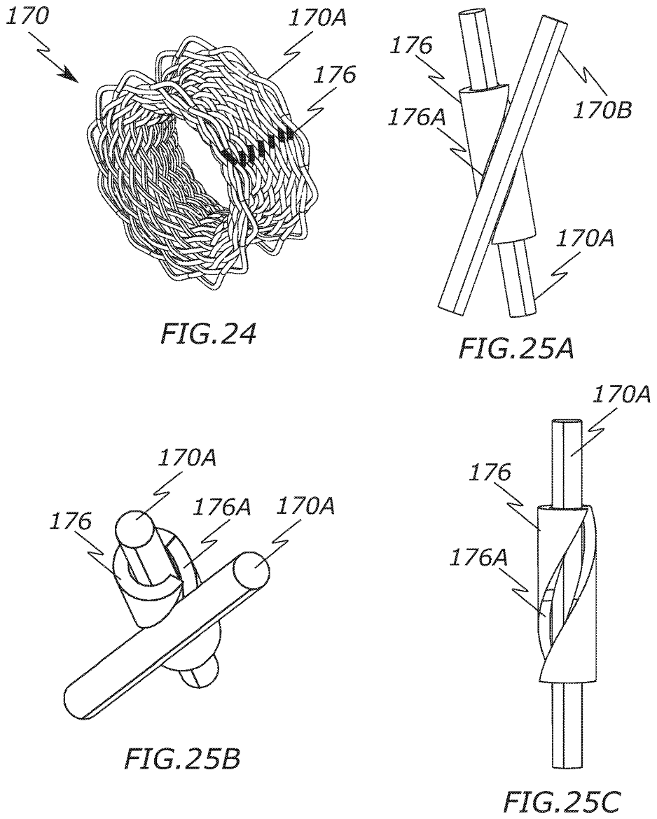

FIGS. 24 and 25A-25C illustrate examples of locking mechanisms configured to selectively resist braid-induced coupling between axial length and diameter in a fully braided shunt stent 170. In one example of a locking mechanism, the shunt stent 170 includes one or a plurality of sleeves 176 mounted on a first braid wire 170A. Each sleeve 176 defines a groove or channel 176A oriented at a designed angle relative to the wire axis and is sized to receive and engage a crossing, second braid wire 170B when the stent 170 reaches a target expanded diameter. Upon alignment during radial expansion, the wire 170B nests in the groove 176A, thereby inhibiting further axial sliding at that crossing and locally “locking” the diameter of the stent 170 near the sleeve 176 without rigidly fixing other regions of the braid. Hence, shunt stent 170 may maintain its expanded shape and particularly its lumen size despite receiving forces that might otherwise affect that diameter (e.g., axial forces).

In use, the braided construct behaves normally during delivery and early expansion, allowing wires 170A, 170B to slide relative to each other to accommodate catheter constraints and tortuosity. As the stent 170 approaches a designed deployed size, the crossing point of wire 170B indexes into the corresponding groove 176A on sleeve 176 and the features engage. This engagement selectively resists changes in axial length and radial diameter at that site, which mitigates the braided structure's typical inverse length-to-diameter relationship and helps hold the stent 170 near a desired lumen size. The engagement strength may be tuned by the groove depth, width, flank angles, and surface finish, as well as by optional spring-like or detent-like features on the sleeve 176.

The locking mechanism may be configured as permanent (e.g., high-depth groove 176A or undercut profile that prevents back-out under physiologic forces) or as overridable/releasable (e.g., shallower groove or ramped entry/exit chamfers) so that a predetermined load, torsion, or balloon expansion can temporarily disengage wire 170B from groove 176A to permit repositioning or re-sizing.

In some examples, the sleeves 176 are distributed circumferentially around the stent 170 (e.g., three, four, five, six, or more evenly spaced). In some examples, the sleeves 176 are form longitudinal/axial rows between proximal and distal ends of the fully braided shunt stent 170. In some examples, the sleeves 176 may be distributed in a plurality of longitudinal/axial rows and there may be a plurality of rows different circumferential positions. In other examples, the sleeves 176 may be distributed in non-linear longitudinal/axial patterns, such as a plurality of helical patterns. These patterns may be adjusted to improve symmetry of the flanged end portions 174, and/or to stabilize the middle portion 172 diameter while allowing limited compliance elsewhere. In some examples, the sleeves 176 may only be positioned in the middle region of the fully braided shunt stent 170, in the flange portions of the fully braided shunt stent 170, or at both but at different locations of each.

The sleeve 176 may be a short tube (e.g., metal hypotube or microtube) laser-cut to form the groove 176A at a specified helix angle corresponding to the target braid angle at the intended location to achieve a desired deployment diameter. Additional features may be cut or formed into the sleeve 176 to increase engagement, including one or more inwardly directed tabs that are shape-set to stand at a slight angle, local serrations, or micro-barbs that improve seating of the crossing wire 170B once captured. In further examples, the crossing wire 170B may include complementary surface geometry (e.g., a locally ground or swaged square-like profile, knurl, or flattened facet) to increase contact area and reduce the likelihood of unintended slippage within the groove 176A.

Alternative implementations may integrate the groove-like feature directly into one of the braid wires rather than using a separate sleeve. For instance, a localized indentation, notch, or channel can be formed into wire 170A by coining, EDM, grinding, electropolishing, or laser ablation so that wire 170B seats into the wire-integrated pocket at the target diameter. In other examples, ridges, bumps, or collars may be welded, brazed, or otherwise bonded onto a braid wire to produce a functionally similar capture geometry for the crossing wire. Any of these locking features may be used alone or in combination with sleeve-based locks, and may be applied to one or both end portions 174 and/or the middle portion 172 to tailor expansion, foreshortening, and fixation behavior for a given anatomy.

In certain examples, the sleeves 176 are radiopaque and/or include radiopaque markers so that the operator can visualize lock engagement under fluoroscopy. The sleeves may be positioned to engage only at the fully deployed state, to create a two-stage deployment (e.g., partial expansion with free braid sliding followed by final locking), or to provide differential locking where end-portion locks engage before middle-portion locks to shape the flanged geometry. Surface coatings (e.g., low-friction polymers on non-engaging faces, higher-friction textures within the groove 176A) may further refine deployment forces and reduce wear at the wire-sleeve interface. The groove 176A may be cut entirely through the sleeve 176 or may extend only partially through the thickness of the sleeve 176. The groove 176A may have a linear shape parallel to a longitudinal axis of the wire 170A, may have a helical shape at least partially around the sleeve 176, or may form other patterns.

Manufacturing methods may include placing the sleeves 176 onto select wires 170A prior to braiding, fixing their axial locations with crimps or micro-bonding, performing the braid, and then heat-setting the stent 170 to define the smaller-diameter middle portion 172 and larger-diameter end portions 174. The groove 176A geometry (angle and depth) is selected to correspond to the local braid angle and to the intended deployed diameter at that axial position. Quality controls may verify that, at the design diameter, the crossing wire 170B reproducibly drops into the groove 176A with a target engagement force window, providing predictable locking behavior across device sizes.

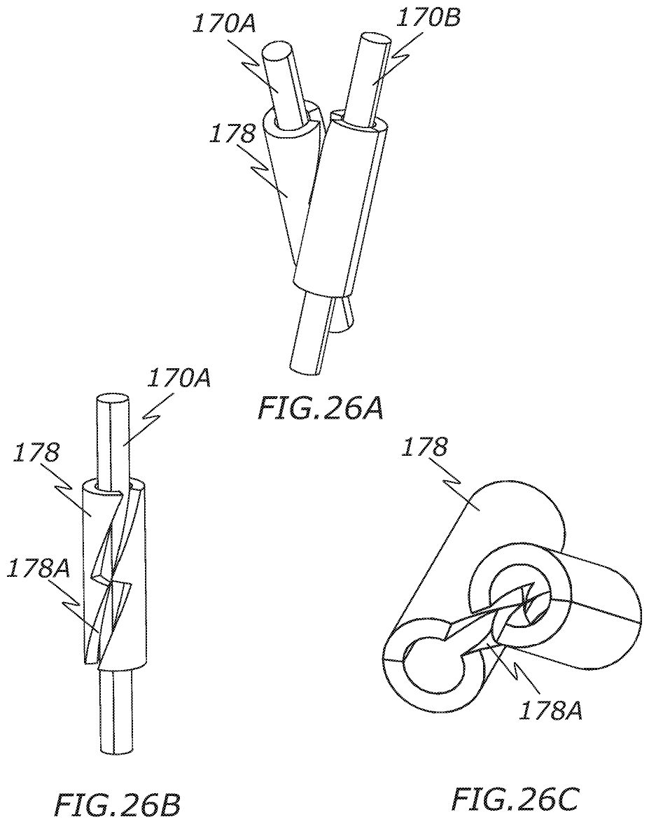

In some examples, a locking mechanism may include one sleeve on each of two overlapping braid wires, rather than a single sleeve engaging a bare crossing wire. FIGS. 26A-26C illustrate one such embodiment in which a first braid wire 170A includes a first sleeve 178 and a second braid wire 170B includes a second sleeve 178, with the sleeves positioned such that they are configured to engage each other at a desired expanded diameter of the fully braided shunt stent 170. In this configuration, locking occurs through sleeve-to-sleeve engagement, rather than direct wire-to-sleeve engagement, which may provide increased engagement area, redundancy, and resistance to relative axial sliding.

Each sleeve 178 may define a groove or channel 178A (similar to channel 176A of sleeve 176), and the channels 178A of the opposing sleeves may be shaped to cooperatively interlock when the stent 170 reaches a target deployed diameter. In one example, the channels 178A have a non-linear, jagged, or lightning-like profile, such that opposing sidewalls of the channels mechanically engage one another when aligned and stop further movement. In an alternative example, each channel 178A may have a helical shape opposite of the other channel 178A, similar to the channel 176A. This interdigitating geometry may resist both axial displacement and rotational movement between the sleeves once engaged, thereby further mitigating braid-induced changes in length and diameter at the locked location.

As with other locking embodiments described herein, the geometry of the channels 178A, including depth, flank angle, waveform amplitude, and pitch of the jagged profile, may be selected to define the expanded shunt stent diameter at which engagement occurs and the force required to disengage the sleeves. In some examples, the interlocking sleeve arrangement provides a permanent lock, while in other examples the channel geometry allows the sleeves 178 to be disengaged upon application of a predetermined axial load, torsional load, or radial expansion force. The dual-sleeve configuration may therefore be tuned to provide either irreversible fixation or controlled overridability, depending on clinical and deployment considerations.

The sleeves 178 may be positioned in the areas identified for sleeve 176, such as at multiple locations around the circumference and/or along the length of the shunt stent 170 to establish a desired locking pattern. For example, multiple sleeve pairs may be circumferentially distributed to promote symmetric expansion of the end portions 174, while one or more sleeve pairs may be positioned along the middle portion 172 to stabilize the lumen diameter. The sleeves 178 may be identical or may differ in channel geometry to provide differential locking behavior at different axial locations of the stent.

Manufacturing of the dual-sleeve embodiment may include placing sleeves 178 onto selected braid wires 170A, 170B prior to braiding, fixing their axial positions, and then braiding and heat-setting the stent 170 so that the sleeves align and interlock at the intended deployed diameter. As with other sleeve-based locking mechanisms, the sleeves 178 may include radiopaque material or markers to facilitate visualization of lock engagement during deployment. The dual-sleeve interlocking approach may provide additional surface contact versus the single sleeve 176 and therefore enhanced robustness, repeatability, and resistance to fatigue-related disengagement while preserving the inherent flexibility and conformability of the braided shunt stent.

In addition to the sleeve-based locking mechanisms described herein, other forms of selective diameter-stabilizing mechanisms may be used with the braided tubular structure. For example, in some embodiments, a sliding or locking suture mechanism may be incorporated into one or more filaments of the braid. Such a mechanism may include a suture, wire, or filament that passes through one or more loops, collars, or eyelets positioned along the braid and is configured to cinch, tighten, or lock when pulled to a user-selected length, thereby resisting subsequent axial movement of the braid filaments once the shunt stent reaches a desired expanded diameter. In other examples, a ratchet-, toothed-, or zip-tie-like locking member may be attached to or integrated with one or more braid wires, such that advancement of the locking element through a corresponding receiving structure allows incremental tightening and prevents reverse motion under physiologic loading. These alternative locking mechanisms may function alone or in combination with sleeve-based locks, and may provide user-adjustable, one-way, or reversible engagement behaviors. As with the sleeve-based mechanisms, such locking features may be positioned in the middle portion of the braid to stabilize the shunt lumen diameter, in one or both end portions to stabilize tissue-engaging regions, or in patterned distributions to achieve desired expansion, foreshortening, and fixation characteristics.

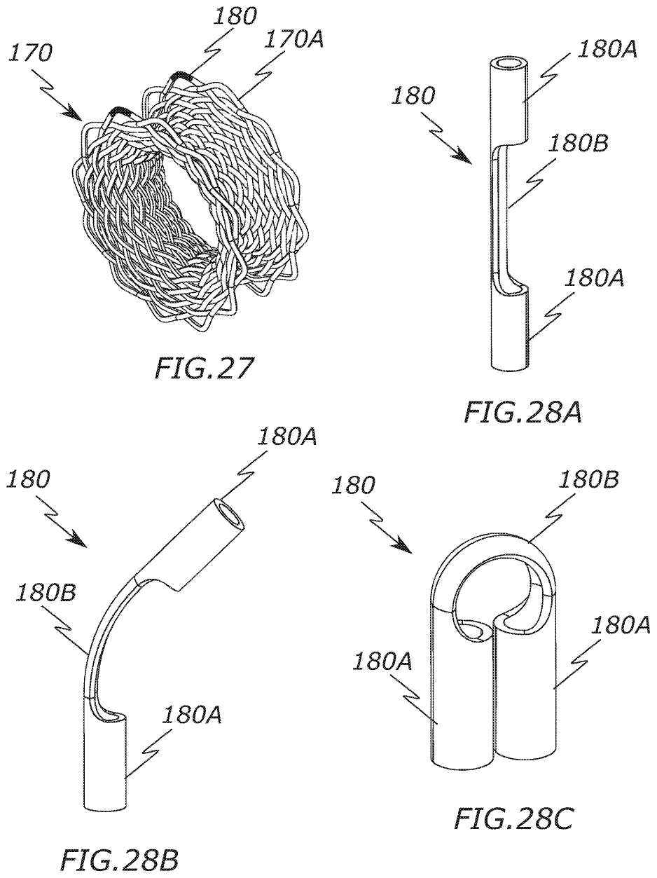

FIGS. 27, 28A, 28B, and 28C illustrate a shunt stent 170 configured with a plurality of discrete hinges 180 positioned on one or both of the proximal and distal end loops of the braided structure. In general, the hinges 180 are arranged at selected braid crossover locations or along loop segments that experience the highest curvature during crimping and re-sheathing. By locally concentrating bending compliance at these points, the hinges 180 allow the end loops to articulate to a more acute angle when the shunt stent 170 is radially constrained on a delivery system, while helping to limit uncontrolled kink formation elsewhere in the braid. In expanded service, the hinges 180 recover toward a low-strain configuration that preserves the intended flange geometry of the end portions and maintains the target inner lumen size of the middle portion 172.

In some examples, each hinge 180 is formed as a short tubular member that includes a laser-cut pattern in a central region to remove a portion of the tube wall and thereby define a compliant bending zone. The hinge tube may be fabricated from a thin-wall metallic hypotube (e.g., nitinol, stainless steel, or cobalt-chromium) or a polymeric microtube with sufficient fatigue resistance under cyclic atrial loading. The compliant region may include a window or slot extending partially or fully through the tube wall, straight or curved struts bridging the window, or a series of axial grooves that reduce section modulus in the bend plane. These patterns are configured so that, under radial crimping, the hinge 180 folds in a controlled manner (see FIGS. 28A-28C), increasing allowable loop deflection while limiting plastic strain in adjacent braid wires 170A.

The hinges may be distributed circumferentially at uniform or non-uniform spacing to shape deployment symmetry and to avoid interference with optional sleeve-based locking features elsewhere on the shunt stent 170 (e.g., on every loop, alternating loops, or other patterns). In some examples, a plurality of hinges 180 are positioned circumferentially around each proximal and/or distal end loop of the shunt stent 170. The hinges may be evenly spaced, such as every 2-5 braid crossover points, or non-uniformly spaced to tailor loop articulation and symmetry during crimping. Hinges 180 may be provided on both proximal and distal end loops, or only on one end, depending on desired delivery profile and deployment behavior. While shown with fully braided shunt stent 170, one or a plurality of hinges 180 may also be used with the braided portion 164 of the shunt stent 160.

The hinge 180 may be coupled to the braided stent framework by locally cutting a braid wire 170A and attaching the hinge between the opposing wire ends. In one arrangement, the hinge 180 has two tubular end segments 180A that telescope over the corresponding trimmed ends of the braid wire 170A, and a middle cut-away region 180B that provides the compliant bend. The tubular end segments 180A may be secured to the wire using laser welding, resistance welding, brazing, crimping, soldering, adhesive bonding, or combinations thereof. Joint geometry can include straight sleeves, swaged collars, or stepped sockets to increase bond length and distribute stress.

Alternative hinge implementations are also contemplated. In some examples, axial or helical grooves are cut directly into a microtube to form the hinge 180, without removing a full window of material. In other examples, non-tubular hinge elements (e.g., stamped leaf-spring links, open-frame bridges, or micro-coil sections) are attached between cut ends of the braid wire 170A to provide localized flexure. In a further alternative, the hinge function is integrated into the braid wire itself by locally reducing the wire diameter (e.g., by centerless grinding, coining, electropolishing, or etching) to create a thin-section region that preferentially bends during crimping. Any of these hinge concepts may be used alone or in combination on the same stent to tailor end-loop articulation, profile during crimping, and expansion behavior upon release.

In operation, the hinges 180 enable the end portions (flanges) of the fully braided shunt stent 170 to collapse more compactly around the delivery mandrel or inner shaft while maintaining braid alignment and minimizing out-of-plane kinks. This can reduce the crimp diameter and delivery profile and may improve re-sheathing reliability if partial deployment repositioning is required. Upon release from the outer sheath, the hinges 180 elastically unfold and allow the end loops to assume the designed expanded geometry, thereby promoting secure tissue engagement on opposing sides of the shunt tract and helping to maintain the intended lumen diameter of the middle portion 172. The hinge features are compatible with the stent's braided construction and, where included, with selective locking mechanisms that stabilize the deployed diameter.

Manufacturing methods may include: (i) placing pre-cut hinge tubes on selected wires prior to braiding and fixing their axial location, then performing the braid and heat-setting; (ii) braiding the shunt stent 170 first, then selectively cutting one or more wires 170A at end-loop positions and splicing hinge members in situ; or (iii) weaving non-tubular hinge elements into the braid during fabrication. Process controls can verify hinge placement, bending torque, and fatigue durability under simulated crimp/expand cycles. Where the device includes semi-porous or non-porous coverings, local windows or flexible patches can be aligned with hinges to preserve articulation without wrinkling the covering film.

Notes on integration with existing description: The features above are intended to complement the previously described fully braided stent architecture, including the middle portion 172 and enlarged end portions 174 that provide a rivet-like effect during tissue engagement, and may be used alongside optional sleeve-based locks that stabilize braid diameter after deployment.

In some examples, the hinge 180 has an overall axial length within an inclusive range of about 0.5 mm to about 3.5 mm, such as from about 0.8 mm to about 2.0 mm, selected to provide localized flexibility while limiting disruption to the surrounding braid geometry. Each tubular end segment 180A may have an axial length within an inclusive range of about 0.15 mm to about 1.2 mm, sufficient to overlap and securely attach to the adjacent ends of the braid wire 170A. The middle compliant region 180B may have an axial length within an inclusive range of about 0.1 mm to about 1.5 mm, and may extend over only a portion of the hinge circumference or around the full circumference depending on the desired bend directionality.

In examples where the hinge 180 includes a cut-away window, slot, or groove-based pattern, the removed wall portion may occupy an angular span within an inclusive range of about 60 degrees to about 300 degrees of the tubular circumference. The remaining wall thickness in the compliant region 180B may be within an inclusive range of about 20% to about 70% of the original tube wall thickness, thereby reducing section modulus while retaining sufficient strength for cyclic loading. Where grooves are used instead of a full window, the grooves may be oriented axially, circumferentially, helically, or in combinations thereof, with groove depths selected to preferentially define a bending plane.

The hinge 180 may be configured to permit bending of the associated braid wire 170A to an acute angle during crimping. In some examples, the hinge allows a local bend angle within an inclusive range of about 30 degrees to about 180 degrees between adjacent wire segments when the shunt stent 170 is crimped onto a delivery device. In certain examples, the hinge permits bending of 60 degrees or greater without inducing permanent deformation of the wire 170A adjacent to the hinge. Upon release from the delivery system, the hinge 180 may elastically recover so that the wire segments return to within about 10 degrees of their pre-crimp orientation, thereby preserving the intended expanded geometry of the end loops.

In some examples, the hinge 180 is fabricated from a metallic hypotube formed from nitinol, stainless steel, cobalt-chromium alloys, titanium alloys, or combinations thereof, selected for fatigue resistance and elastic recoverability under repeated crimping and cardiac cycles. In other examples, the hinge 180 may be formed from a polymeric material, such as polyether ether ketone (PEEK), polyimide, nylon, or polyurethane, optionally reinforced with fibers or fillers. Where dissimilar metals are used between the hinge 180 and the braid wire 170A, the attachment method may be selected to limit galvanic corrosion, including laser welding with suitable filler material or use of an intermediate sleeve.

In examples where the hinge 180 is attached to cut ends of a braid wire 170A, the tubular end segments 180A may have an inner diameter sized to create an interference fit, slip fit, or adhesive-bonding fit with the wire ends. The overlap length between each tubular end segment 180A and the wire 170A may be within an inclusive range of about 0.1 mm to about 1.0 mm, and circumferential welds, spot welds, or longitudinal seam welds may be used individually or in combination. In other examples, mechanical attachment may be achieved using crimping, swaging, or micro-barb features formed on the inner surface of the tubular end segments 180A.

Alternative hinge implementations are also contemplated in which the hinge function is integrated directly into the braid wire 170A. In such examples, a local diameter reduction of the wire 170A may be provided over an axial length within an inclusive range of about 0.2 mm to about 2.0 mm, reducing the cross-sectional area by about 20% to about 70% relative to adjacent wire portions. This reduced-diameter region may be formed by centerless grinding, chemical etching, laser ablation, or coining, and may function as a compliant hinge region without requiring a separate tubular component.

Where desired, one or more hinges 180 may include radiopaque material or markers, such as platinum-iridium bands, tantalum markers, or radiopaque coatings, to facilitate fluoroscopic visualization of hinge orientation and end-loop articulation during delivery and deployment.

Any of the features described in the embodiments of this application should be understood to be, optionally, combined with each other. Hence, while different example features are described separately, they should be understood to be potentially incorporated with each other.

1-110. (canceled)

111. A method of creating a shunt between a left atrial appendage (LAA) and a pulmonary vein (PV), the method comprising:
advancing a catheter-based instrument into a heart;
forming an opening that traverses a wall of the left atrial appendage and a wall of the pulmonary vein; and,
establishing a flow path between the pulmonary vein and the left atrial appendage that permits blood to pass into and out of the left atrial appendage.

112. The method of claim 111, wherein advancing the catheter-based instrument comprises advancing an outer sheath over a guidewire toward the left atrial appendage and orienting a distal opening of the outer sheath toward a selected pulmonary vein.

113. The method of claim 112, wherein the outer sheath includes a pre-formed distal curve, a steerable mechanism to direct the distal opening toward the PV, or both.

114. The method of claim 111, wherein forming the opening comprises delivering radiofrequency energy via an electrode at a distal tip of an inner shaft advanced through an outer sheath.

115. A method claim 111, wherein formation of the opening includes apposing the LAA wall and the PV wall with an instrument deployed through a sheath.

116. The method of claim 111, wherein establishing the flow path comprises deploying a shunt stent across the opening to define a shunt lumen between the left atrial appendage and the pulmonary vein.

117. The method of claim 116, wherein the shunt stent is self-expanding and is released by withdrawing an outer shaft from over an inner shaft.

118. The method of claim 116, wherein the shunt stent includes a strut portion and at least one braided portion that foreshortens upon expansion to engage tissue on opposite sides of the opening.

119. The method of claim 118, wherein the strut portion is formed from a laser-cut tubular member and the braided portion comprises at least one of metallic and polymeric filaments.

120. The method of claim 116, wherein the shunt stent is a braided construct having a smaller-diameter middle portion and enlarged end portions that provide rivet-like tissue engagement.

121. A method of creating a shunt between a left atrial appendage (LAA) and a pulmonary vein (PV), the method comprising:
advancing a catheter-based instrument into a heart;
forming an opening that traverses a wall of the left atrial appendage and a wall of the pulmonary vein;
establishing a flow path between the pulmonary vein and the left atrial appendage that permits blood to pass into and out of the left atrial appendage;
wherein the shunt stent is a fully braided construct having a smaller-diameter middle portion and enlarged end portions that provide rivet-like tissue engagement;
wherein establishing the flow path comprises deploying a shunt stent across the opening to define a shunt lumen between the left atrial appendage and the pulmonary vein; and,
further comprising crimping the end portions with localized hinges positioned along end loops to permit acute-angle bending during loading onto a delivery device and elastic recovery upon deployment.

122. The method of claim 121, wherein each hinge includes tubular end segments attached to cut ends of a braid wire and a middle compliant region formed by a laser-cut pattern.

123. The method of claim 121, wherein the hinge provides a bend angle between about 30 degrees and about 180 degrees during crimping and elastic recovery to within about 10 degrees of a pre-crimp orientation.

124. A method of creating a shunt between a left atrial appendage (LAA) and a pulmonary vein (PV), the method comprising:
advancing a catheter-based instrument into a heart;
forming an opening that traverses a wall of the left atrial appendage and a wall of the pulmonary vein;
establishing a flow path between the pulmonary vein and the left atrial appendage that permits blood to pass into and out of the left atrial appendage; and
further comprising performing a contrast washout assessment of the LAA after establishing the flow path to evaluate shunt effectiveness.

125. The method of claim 124, wherein advancing the catheter-based instrument comprises advancing an outer sheath over a guidewire toward the left atrial appendage and orienting a distal opening of the outer sheath toward a selected pulmonary vein.

126. The method of claim 125, wherein the outer sheath includes at least one of a pre-formed distal curve, and a steerable mechanism to direct the distal opening toward the PV.

127. The method of claim 124, wherein forming the opening comprises delivering radiofrequency energy via an electrode at a distal tip of an inner shaft advanced through an outer sheath.

128. A method claim 124, wherein formation of the opening includes apposing the LAA wall and the PV wall with an instrument deployed through a sheath.

129. The method of claim 124, wherein establishing the flow path comprises deploying a shunt stent across the opening to define a shunt lumen between the left atrial appendage and the pulmonary vein.

130. The method of claim 129, wherein the shunt stent is self-expanding and is released by withdrawing an outer shaft from over an inner shaft.

131. The method of claim 129, wherein the shunt stent includes a strut portion and at least one portion that foreshortens upon expansion to engage tissue on opposite sides of the opening.

132. The method of claim 129, wherein the shunt stent is a braided construct having a smaller-diameter middle portion and enlarged end portions that provide rivet-like tissue engagement.

133. A method of creating a shunt between a left atrial appendage (LAA) and a pulmonary vein (PV), the method comprising:
advancing a catheter-based instrument into a heart;
forming an opening that traverses a wall of the left atrial appendage and a wall of the pulmonary vein;
establishing a flow path between the pulmonary vein and the left atrial appendage that permits blood to pass into and out of the left atrial appendage, and
apposing the LAA wall and the PV wall prior to or during formation of the opening using a support instrument advanced or retracted through a sheath.

134. The method of claim 133, wherein advancing the catheter-based instrument comprises advancing an outer sheath over a guidewire toward the left atrial appendage and orienting a distal opening of the outer sheath toward a selected pulmonary vein.

135. The method of claim 134, wherein the outer sheath includes at least one of a pre-formed distal curve and a steerable mechanism to direct the distal opening toward the PV.

136. The method of claim 133, wherein forming the opening comprises delivering radiofrequency energy via an electrode at a distal tip of an inner shaft advanced through an outer sheath.

137. The method of claim 133, wherein establishing the flow path comprises deploying a shunt stent across the opening to define a shunt lumen between the left atrial appendage and the pulmonary vein.

138. The method of claim 137, wherein the shunt stent is self-expanding and is released by withdrawing an outer shaft from over an inner shaft.

139. The method of claim 137, wherein the shunt stent includes a strut portion and at least one portion that foreshortens upon expansion to engage tissue on opposite sides of the opening.

140. The method of claim 137, wherein the shunt stent is a braided construct having a smaller-diameter middle portion and enlarged end portions that provide rivet-like tissue engagement.
